(19) United States
(12) Patent Application Publication
(10) Pub. No.: US 2024/0293289 A1
(43) Pub. Date: Sep. 5, 2024

(54) Multifunctional Dental Adhesives and Uses Thereof

(71) Applicant: University of Maryland, Baltimore, Baltimore, MD (US)

(72) Inventors: Mary Anne S. Melo, Hanover, MD (US); Michael D. Weir, Silver Spring, MD (US); Lamia Sami Mokeem, Jessup, MD (US)

(73) Assignee: University of Maryland, Baltimore, Baltimore, MD (US)

(21) Appl. No.: 18/593,423

(22) Filed: Mar. 1, 2024

Related U.S. Application Data
(60) Provisional application No. 63/449,334, filed on Mar. 2, 2023.

(51) Int. Cl. A61K 6/69; A61K 6/30; A61K 6/71; A61K 6/76; A61K 31/155; A61K 31/695

(52) U.S. Cl. CPC A61K 6/69; A61K 6/30; A61K 6/71; A61K 6/76; A61K 31/155; A61K 31/695

(57) ABSTRACT

Provided herein are dental adhesives that contain a dental adhesive material and a multifunctional magnetic platform. The multifunctional magnetic platform has a core-shell structure. The core includes magnetic microparticles and an antibiotic with the core capped by an antibacterial silane coating. Also provided are methods utilizing the dental adhesives for increasing longevity of a dental restoration and for restoring a tooth in a subject.

### CROSS-REFERENCE TO RELATED APPLICATIONS

This non-provisional patent application claims benefit of priority under 35 U.S.C. §119(e) of provisional application U.S. Ser. No. 63/449,334, filed Mar. 2, 2023, the entirety of which is hereby incorporated by reference.

### STATEMENT OF FEDERALLY SPONSORED RESEARCH AND DEVELOPMENT

This invention was made with government support under grant number DE030562 awarded by the National Institutes of Health. The government has certain rights in the invention.

### BACKGROUND OF THE INVENTION

### Field of the Invention

The present invention relates to the fields of dentistry, oral diseases, and drug delivery systems. More particularly, the present invention relates to microemulsions as magnetic responsive nanoplatforms for the delivery of formulations to pathogenic microbes in the oral environment during photodynamic therapy.

### Description of the Related Art

The longevity of resin composite dental restorations relies heavily on the effectiveness of dental adhesives in bonding the tooth-colored material to the dentin, the inner part of the tooth (1,2). This bonding interface forms a hybrid layer, combining dental adhesive with dentin components like collagen, water, and hydroxyapatite crystals. However, this interface is subjected to mechanical stress, temperature changes, and bacterial acid attacks in the mouth. Evidence indicates this interface weakens over time, leading to poor sealing between the restorative material and dentin (3). This can result in issues like microleakage, tooth sensitivity, and cavities around restorations, necessitating costly replacement (4).

Superparamagnetic iron oxide nanoparticles (SPIONs) have shown promise in dentistry as reinforcing fillers that can exhibit antibacterial properties and strengthen resins (5). These particles, in particular, have garnered significant interest in dental adhesion due to their unique properties, such as enhanced adhesive strength and the ability to deliver drugs by magnetic motion, making them a highly desired material for advancing dental restoration (6).

Core-shell technology, where superparamagnetic iron oxide nanoparticles are encapsulated, can mitigate these particles' aggregation and dark coloration (7). The controlled release of chlorhexidine (CHX) from core-shell particles could be beneficial to protect collagen fibrils from degradation (8). CHX has dual benefits: it acts as an antibacterial agent against dental plaque and inhibits collagen-degrading enzymes (9).

Particles like superparamagnetic iron oxide nanoparticles require surface modification through silanization to enhance bonding performance (10). This chemical modification should not compromise the particle's properties, including saturation magnetization (11). For dental applications, organosilicons play a crucial role in creating a strong interphase between filler and resin, contributing to the strength and durability of dental bonds (12). Furthermore, organosilicon quaternary ammonium salt monomers have long-lasting antibacterial effects (13).

The prior art is deficient in multifunctional dental adhesives. The present invention fulfills this long-standing need and desire in the art.

### SUMMARY OF THE INVENTION

The present invention is directed to a biomedical composition for dental restoration. The biomedical composition has a plurality of magnetic microparticles, an antibiotic and a mesoporous silica encapsulating the plurality of magnetic microparticles and the antibiotic with an antibacterial shell disposed therearound.

The present invention is further directed to a dental formulation for a dental bonding restoration. The dental formulation has a dental adhesive material and the biomedical composition described herein incorporated therein.

The present invention is directed further to a method for increasing longevity of a dental restoration in a subject. In this method, the dental formulation described herein is prepared and is applied to an area of interest in an oral cavity of the subject. The dental formulation is bonded to dentin in the area of interest, where the magnetic microparticles, the antibiotic and the antibacterial shell of the biomedical composition incorporated therein increase the longevity of the dental restoration in the subject.

The present invention is directed further still to a dental adhesive. The dental adhesive has a biomedical adhesive and a magnetic core-shell platform incorporated therein, where the dental adhesive is pharmacologically effective for a tooth restoration.

The present invention is directed further still to a method for restoring a tooth in a subject in need thereof. In this method, a dental adhesive comprising a dental adhesive material and containing a multifunctional magnetic platform therein is formulated. The tooth is prepared for restoration, the dental adhesive is applied to a dentin layer on the tooth and the tooth is restored.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B show a graphic representation of the multifunctional platform (FIG. 1A) and a restored tooth with interface composite/tooth (FIG. 1B) (denoted CHX@SiQuac@Fe₃O₄@m-SiO₂). Its composition comprises Fe₃O₄ microparticles and CHX encapsulated with mesoporous silica, capped by an antibacterial SiQuac, and its incorporation into dental adhesives formulations. FIG. 1B shows a restored tooth with interface composite/tooth with a detailed description of properties/effects that the multifunctional magnetic platform offers: (1) filler reinforcement and motion, (2) a combinatorial antibacterial effect, and (3) an antibond degradation strategy via protease inhibitor.

FIGS. 2A-2J are TEM images of Fe₃O₄ showing aggregated cubic particles (FIGS. 2A-2B), the histogram representing the particle's size with a non-normal distribution with a mean size of 191±30 nm (FIG. 2C). Fe₃O₄@m-SiO₂ TEM images showed irregular morphology of cubic and spherical particles with a uniform thin shell. Less aggregation was noticeable in Fe₃O₄@m-SiO₂ particles (FIGS. 2D-2E). The core@shell particle's size averaged 172±41 nm, while the shell thickness was 15.4±5 nm (FIGS. 2F, 21). SEM images presented CHX@SiQuac@Fe₃O₄@m-SiO₂ as cubical rhomboidal particles with semismooth surfaces in ×500 (FIG. 2G), ×5000 (FIG. 2H) and shell thickness (FIG. 21). Elemental analysis (EDS) of CHX@SiQuac@Fe₃O₄@m-SiO₂ showed the dominant presence of C by around 46 wt % (FIG. 2J).

FIGS. 3A-3G characterize the CHX@SiQuac@Fe₃O₄@m-SiO₂ platform. FIG. 3A shows the N₂ adsorption-desorption isotherms of Fe₃O₄@m-SiO₂ and SiQac@Fe₃O₄@m-SiO₂ correspondingly to pore volume. Barret-Joyner-Halenda (BJH) pore size and volume analysis were used to calculate the size distribution of the mesoporous shell (FIG. 3B). The table below depicts the surface area, pore volume, and particle size calculated with BET and BJH methods.

| | Surface | | | | |
|---|---|---|---|---|---|
| | Area | Pore Volume (m²/g) | | Pore size (nm) | |
| | (m²/g) | BJH | BJH | BJH | BJH |
| Sample | (BET) | Adsorption | Desorption | Adsorption | Desorption |
| Fe₃O₄@m-SiO₂ | 32.706 | 0.083 | 0.088 | 13.98 | 13.035 |
| SiQuac@Fe₃O₄@m-SiO₂ | 68.704 | 0.208 | 0.220 | 16.04 | 13.28 |

FIG. 3C indicates the TGA and DTA analysis of the core-shelled and drug-loaded silanized nanoparticles to determine the composition/mass loss over the temperature range and the endo/exothermic event temperature to show the phase transitions. FIG. 3D indicates the CHX cumulative release measured over time using UV-vis spectroscopy, while FIG. 3E indicates the CHX cumulative release analyzed via HPLC. FIG. 3F illustrates the inhibition of MMP-9 by CHX, showing that substantial inhibition can be achieved with less than 1 mg/mL of CHX. FIG. 3G presents the release profile of CHX from the tested formulations, both with (CHX@SiQuac@Fe₃O₄@m-SiO₂) and without silane (CHX@Fe₃O₄@m-SiO₂) under neutral and acidic conditions over the initial 24 h period. The released amounts exceeded 1 mg/mL, suggesting that CHX@SiQuac@Fe₃O₄@m-SiO₂ can potentially release CHX at concentrations effective for inhibition.

FIGS. 4A-4L show microscopic images of the dental pulp stem cells (DPSC). FIG. 4A represents the 24 h of growth, FIG. 4B represents 72 h of growth and FIG. 4C represents 24 h of growth after adding the particles. FIG. 4D is a fluorescence microscopy image using Live/Dead staining it after 24 h of cells exposure to CHX@SiQuac@Fe₃O₄@m-SiO₂. FIG. 4E shows the viability of DPSC. FIG. 4F shows the viability of human gingival fibroblasts (HGF). For both graphs, different lowercase letters represent the statistical significance difference among CHX@Fe₃O₄@m-SiO₂ groups(p<0.05), and different uppercase letters represent the statistical significance difference among CHX@SiQuac@Fe₃O₄@m-SiO₂ groups (p<0.05). Microscopic images of the HGF are indicated below. FIG. 4G shows HGF after 24 h of growth, FIG. 4H shows HGF after 72 h of growth and FIG. 4I shows HGF after 24 h after adding the particles. FIG. 4J is a fluorescence microscopy image using Live/Dead staining kit after 24 h of cells exposure to the particles. FIG. 4K shows the reduction percentage of S. mutans in the planktonic count, and FIG. 4L shows the reduction percentage of this bacteria in biofilm after exposure to CHX@Fe₃O₄@m-SiO₂ and CHX@SiQuac@Fe₃O₄@m-SiO₂ at different concentrations. Lower case letters indicate a statistically significant difference among CHX@SiQuac@Fe₃O₄@m-SiO₂. while upper case letters represent a statistically significant difference among containing CHX@Fe₃O₄@m-SiO₂ (p<0.5)

FIGS. 5A-5J show a comprehensive analysis of the physicochemical properties of dental adhesives with different concentrations of CHX@Fe₃O₄@m-SiO₂ and CHX@SiQuac@Fe₃O₄@m-SiO₂. FIGS. 5A-B illustrate the molecular structures of the main dental adhesive monomers, Hydroxyethyl methacrylate (HEMA) and Bisphenol A-glycidyl methacrylate (Bis-GMA). FIG. 5C is a bar graph showing the degree of conversion (%) for dental adhesives with varying concentrations of CHX@Fe₃O₄@m-SiO₂ and CHX@SiQuac@Fe₃O₄@m-SiO₂ from 2 to 5 wt. %, in comparison to an untreated control. FIG. 5D displays the contact angle between water drops and the polymerized adhesive samples and the corresponding formed droplets are shown in image FIG. 5F. FIGS. 5E and 5G show the contact angle between the unpolymerized adhesives and dentin and its corresponding formed droplets. The results of surface charge density are represented in image FIG. 5H. The results of water sorption (SP) and water solubility (WS) are depicted in images FIGS. 51-J. Different letters represent a statistically significant difference among groups (p<0.05).

FIGS. 6A-6D show ultimate tensile strength (UTS) represented in MPa (FIG. 6A) and microshear bond strength (μ-SBS) between the formulated adhesives and human dentin (FIG. 6B). FIG. 6C shows the results of μ-TBS at two different time points, immediately and after aging. FIG. 6D shows the fracture pattern analysis results. Different uppercase letters indicate statistically significant group differences (p<0.05). Different lowercase letters indicate statistically significant differences within each group comparing immediate and long-term analysis (p<0.05).

FIGS. 7A-7Q show antibacterial and cytotoxicity of CHX@SiQuac@Fe₃O₄@m-SiO₂ in the dental adhesive on S. mutans. FIG. 7A is an immediate CFU Counting Assay that demonstrates the viability of S. mutans after exposure to formulated dental adhesives. FIG. 7B shows the immediate metabolic activity of S. mutans biofilms grown over the tested materials. FIG. 7C shows Live/Dead staining displaying S. mutans biofilms on adhesive samples from various groups. FIG. 7D shows SEM imaging of representative images of the S. mutans biofilm. FIG. 7E shows a CFU assay comparison that compares bacterial reduction in immediate vs. aged adhesive samples using the CFU assay. FIG. 7F shows metabolic activity over time whichontrasts immediate and long-term metabolic activity of S. mutans. FIG. 7G shows an aged sample biofilm showing live/dead staining of biofilms on aged adhesive samples. FIG. 7H shows an human gingival fibroblast (HGF) viability test that assesses the viability of human gingival fibroblast cells against different adhesive sample elutes. FIG. 71 is an immediate human gingival fibroblast cell observation showing a microscopic view of human gingival fibroblast cells at immediate seeding. FIG. 7J shows human gingival fibroblast growth after 72 hours representing human gingival fibroblast cell growth 72 hours post-seeding. FIG. 7K shows human gingival fibroblast exposure to adhesive elutes (24 Hours) with a microscopic view of HGF cells after 24-hour exposure to adhesive elutes. FIG. 7L shows the Live/Dead staining of human gingival fibroblast Cells. FIG. 7M shows dental pulp stem cells (DPSC) viability illustrating the viability of dental pulp stem cells. FIGS. 7N-7Q illustrate dental pulp stem cells maturation and multiplication detailing the process of cell maturation and multiplication of dental pulp stem cells. FIGS. 7P-7Q show dental pulp stem cells exposure and analysis that showcase dental pulp stem cells following 24-hour exposure to adhesive elutes, observed under magnification and fluorescence microscopy. Uppercase letters denote significant differences among groups within the same analysis period (immediate or long-term), with p<0.05 indicating significance. Lowercase letters indicate significant differences within each group when comparing immediate and long-term results (p<0.05).

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, “biomedical composition”, “magnetic core-shell platform”, and “multifunctional magnetic platform” are used interchangeably.

As used herein, “biomedical adhesive material” and “dental adhesive material” are used interchangeably.

As used herein, “pharmacologically effective” refers to an amount or concentration of the dental adhesive and/or the multifunctional magnetic platform contained therein that results in an increase in an interfacial bond formed between the dental adhesive and a dentin layer on a tooth being restored. A person having ordinary skill in this art would understand that the pharmacologically effective amount may improve the stability of the bond and increase resistance to degradation thereof and may comprise an antibacterial efficacy for the subject's restored tooth, but may not completely eliminate the problems present with dental adhesives known and standard in the art.

As used herein, the term “subject” refers to any recipient of a dental restoration utilizing the multifunctional magnetic platforms and dental adhesives provided herein.

In one embodiment of the present invention, there is provided a biomedical composition for dental restoration, comprising a plurality of magnetic microparticles; an antibiotic; and a mesoporous silica encapsulating the plurality of magnetic microparticles and the antibiotic with an antibacterial shell disposed therearound.

In this embodiment, the plurality of magnetic microparticles may comprise iron oxide Fe₃O₄. Further in this embodiment, the antibiotic may be chlorhexidine. In addition, the antibacterial shell may be an antibacterial quaternary ammonium silane. Further in this embodiment, the biomedical composition may have a formula CHX@SiQuac@Fe₃O₄@m-SiO₂.

In another embodiment of the present invention, there is provided a dental formulation for a dental bonding restoration, comprising a dental adhesive material; and the biomedical composition as described supra incorporated therein.

In yet another embodiment of the present invention, there is provided a method for increasing longevity of a dental restoration in a subject, comprising preparing the dental formulation as described supra, applying the dental formulation to an area of interest in an oral cavity of the subject; and bonding the dental formulation to dentin in the area of interest, wherein the magnetic microparticles, the antibiotic and the antibacterial shell of the biomedical composition incorporated therein increase the longevity of the dental restoration in the subject. In this embodiment the biomedical composition may strengthen an interfacial bond between the dental adhesive and the dentin.

In yet another embodiment of the present invention, there is provided a dental adhesive, comprising a biomedical adhesive material; and a magnetic core-shell platform incorporated therein, the dental adhesive pharmacologically effective for a tooth restoration.

In this embodiment, the magnetic core-shell platform may comprise a core of a plurality of magnetic microparticles doped with an antibiotic and coated with a mesoporous silane shell that is functionalized with an antibacterial agent.

In an aspect of this embodiment, the plurality of magnetic microparticles may be iron oxide Fe₃O₄. In another aspect, the plurality of magnetic microparticles may be doped with chlorhexidine. In yet another aspect, the antibacterial agent may be an antibacterial quaternary ammonium silane. In this embodiment and all aspects thereof, the magnetic core-shell platform may have a formula of CHX@SiQuac@Fe₃O₄@m-SiO₂.

In yet another embodiment of the present invention, there is provided a method for restoring a tooth in a subject in need thereof, comprising formulating a dental adhesive comprising a dental adhesive material and containing a multifunctional magnetic platform therein; preparing the tooth for restoration; applying the dental adhesive to a dentin layer on the tooth; and restoring the tooth.

In this embodiment, the multifunctional magnetic platform may have a core-shell structure comprising a core formed of a plurality of magnetic microparticles and an antibiotic doping agent; and an antibacterial silane shell coating the core. In an aspect thereof, the core may be formed of a plurality of iron oxide Fe₃O₄ microparticles and doped with chlorhexidine. In another aspect, the antibacterial silane shell may be a mesoporous silane comprising an antibacterial quaternary ammonium silane. In this embodiment and aspects thereof, the multifunctional magnetic platform has a core-shell structure of CHX@SiQuac@Fe₃O₄@m-SiO₂. Also in this embodiment and aspects thereof, the multifunctional magnetic platform contained in the dental adhesive upon forming an interfacial hybrid bond with the dentin layer during restoration may increase longevity thereof via increasing bonding stability via magnetic interactions; resisting degradation of the hybrid bond by matrix metalloproteinases; or inhibiting bacterial growth; or a combination thereof.

Provided herein are the synthesis, characterization, and assessments of a multifunctional magnetic platform incorporated into a dental adhesive to provide 1) filler reinforcement and magnetic motion, 2) a combinatorial antibacterial effect, and 3) an anti-bond degradation strategy via protease inhibitor such as matrix metalloproteinases. This multifunctional platform, denoted CHX@SiQuac@Fe₃O₄@m-SiO₂, comprises Fe₃O₄ microparticles and chlorhexidine (CHX) encapsulated within the mesoporous silica, which is capped by an antibacterial quaternary ammonium silane (SiQuac).

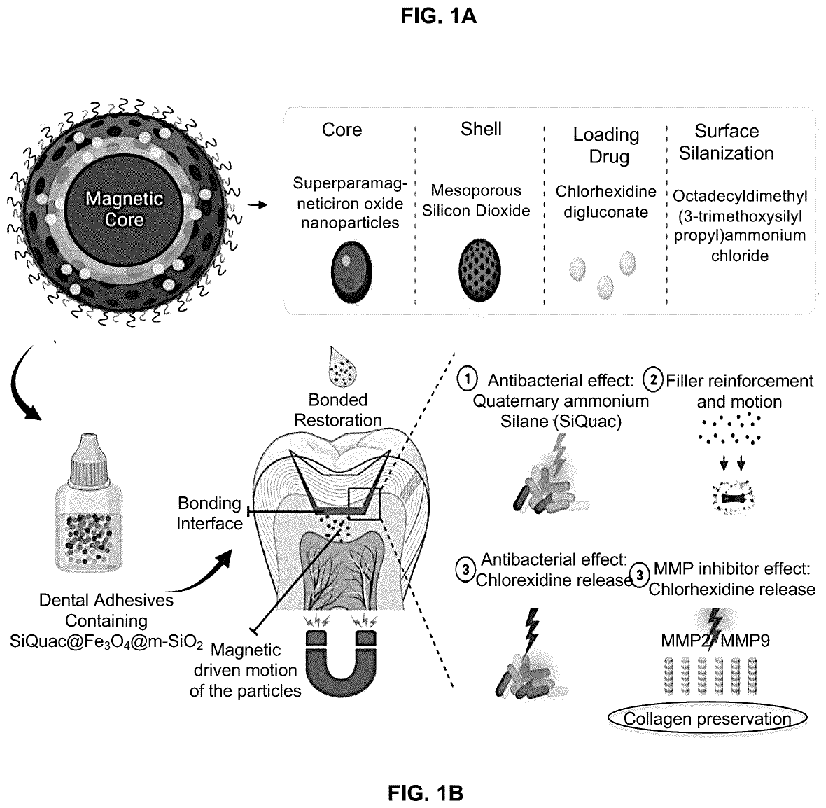

This combinatorial strategy of incorporating silane-coated magnetic core-shell particles, doped with chlorhexidine and functionalized with an antibacterial agent enables the platform to simultaneously target bacterial inhibition, stability of the hybrid layer, and filler reinforcement. The resulting dental adhesive with CHX@SiQuac@Fe₃O₄@m-SiO₂ offers multiple benefits, including bacterial inhibition, bonding stability of the hybrid layer, resistance to degradation, and improved filler infiltration through magnetic motion (FIGS. 1A-1B).

The present invention addresses the critical issue of adhesive interfacial breakdown, which significantly reduces the service life of composite restorations. The multifunctional platform is effective in several aspects. The silanization process enhances the specific surface area and pore size contributing to the improved performance of the material. CHX loading into the framework exhibits increased cumulative release thereof and MMP inhibition, further enhancing the antibacterial and anti-MMP activities without affecting human cell viability.

Also provided herein are methods for increasing longevity of a dental restoration in a subject and for restoring a tooth in a subject in need thereof utilizing the dental adhesives containing the microfunctional magnetic platform described herein. One of ordinary skill in the art is well-able to determine a pharmacologically effective amount of the dental adhesives provided herein for use in a dental restoration procedure or other procedure requiring the same.

The following examples are given for the purpose of illustrating various embodiments of the invention and are not meant to limit the present invention in any fashion.

### EXAMPLE 1

### Methods and Materials

Synthesis of Fe₃O₄@m-SiO₂

The mesoporous silica oxide shell with iron oxide core (Fe₃O₄@m-SiO₂) was synthesized by a sol-gel method (14). Chemicals, including Iron (II,III) oxide nanopowder, TEOS, CHX, dichloromethane, N-propylamine, cyclohexane, and NaOH were procured from Sigma-Aldrich, Saint Louis, MO, USA. Cetyltrimethylammonium bromide CTAB was sourced from EMD Millipore Crop, Burlington, MA, USA, and SiQuac from Gelest Inc, Morrisville, PA, USA. Briefly, 0.007 g of Fe₃O₄, 0.0029 g of CTAB, and 116 μL of TEOS at 20% were dissolved in 5 mL of distilled water with sonication at 40% power, 24 KHz frequency, 37° C. in pulse mode (10 seconds on/10 seconds off) for 80 min. The pH was adjusted to 8 by adding 0.1 M NaOH, and the solution was sonicated again following the previous parameters (66). The solution was kept for 20 h at room temperature. The purification was performed via sonication (same parameters, 12 min), centrifugation (7,000 RCF for 15 mins at 23° C.), and removal of the supernatant. The sedimented particles were redispersed ten times with 25 mL of ethanol and four times with 25 ml of water. The particles were dispersed in 5 ml of water and vacuum dried at 75° C. overnight.

Chlorhexidine Loading into Fe₃O₄@m-SiO₂

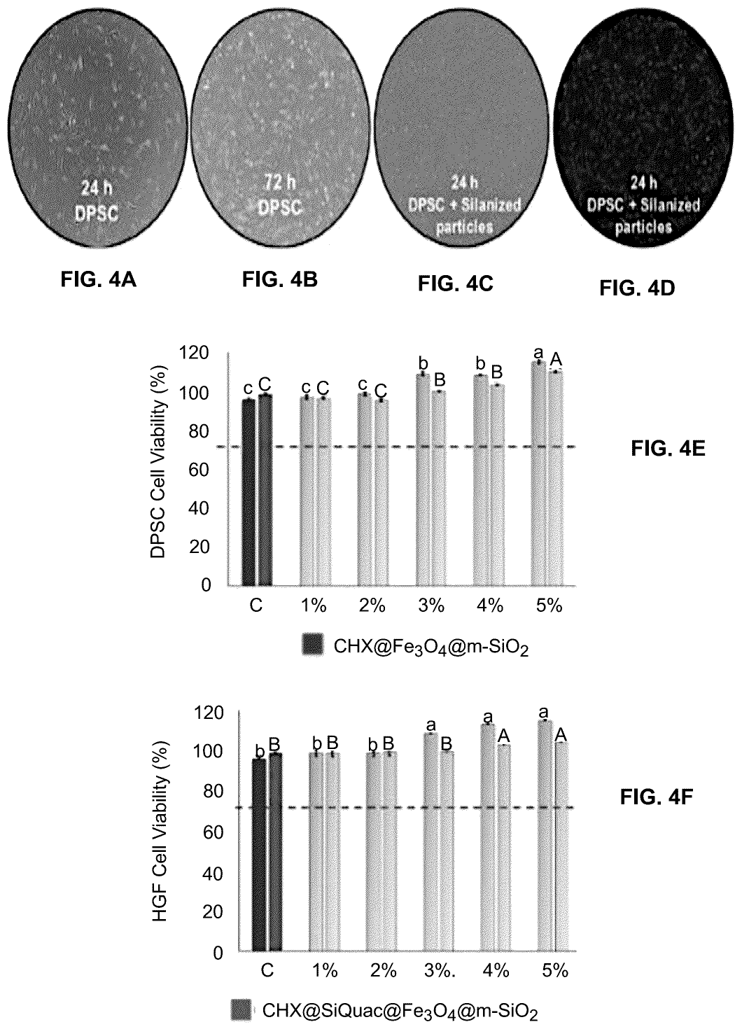

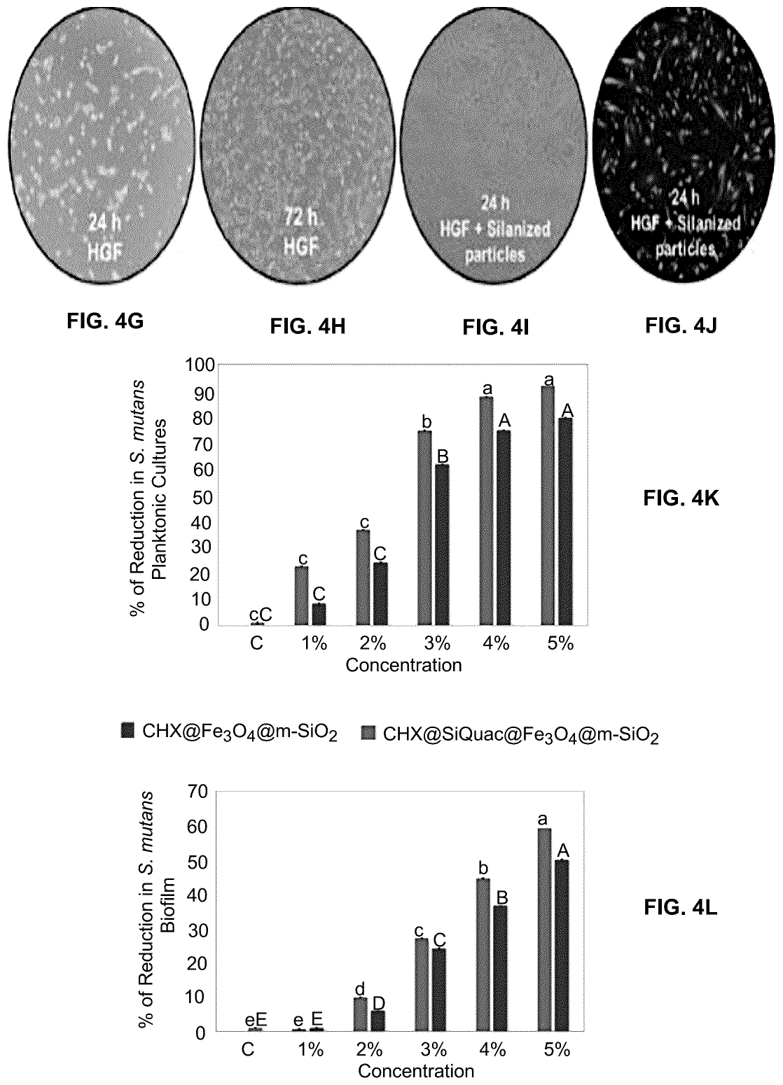

Chlorhexidine (CHX) and Fe₃O₄@m-SiO₂ (50 mg each) were dissolved in 5 mL of dichloromethane by sonication for 12 min, followed by shaking at 100 RPM at room temperature for 24 h. The particles were separated by centrifugation at 7,000 RCF for 10 min at 25° C. The supernatant was preserved to obtain the drug loading. The particles were rinsed with ethanol and water and vacuum dried at 75° C. overnight (FIG. 4E) (15).

Surface Silanization with SiQuac

To functionalize the CHX@Fe₃O₄@m-SiO₂, these particles (2 g) were added to a mix of octadecyl-dimethyl (3-trimethoxysilylpropyl) ammonium chloride (SiQuac) (0.08 mL), n-propylamine (0.04 mL), and cyclohexane (3 mL) (16). The mixture was stirred for 2 h at room temperature. The solution was placed into a rotary evaporator at 90° C. for 1 h, and the powder was dried at 80° C. for 1 h. Some of the CHX@Fe₃O₄@m-SiO₂ was kept without silanization as a control group.

### Characterization of Synthesized Particles

Fe₃O₄@m-SiO₂ and CHX@SiQuac@Fe₃O₄@m-SiO₂ were characterized via transmission electron microscopy (TEM) and scanning electron microscopy (SEM). The images were acquired from 20,000X to 40,000X magnification and 12,500 KV for TEM and 500X to 5,000X magnification with 3.5 kV for SEM. Energy-dispersive X-ray analysis spectroscope (EDS) was used with TEM. The particles were analyzed for surface area and pore size distribution using N₂ adsorption-desorption isotherms. Before the analysis, the particles were kept in a vacuum at 100° C. for 8 h to remove the adsorbed gas. X-ray diffraction (XRD) was used with Cu-Kα radiation (λ=1.54 Å), 40 kV, and 40 mA. The powders were also analyzed with attenuated total reflectance-Fourier transform infrared (ATR/FT-IR) spectroscopy with a diamond-ZnSe crystal. The spectra were acquired from 4000 to 550 cm⁻¹, with a resolution of 0.5 cm⁻¹, and 4 scans/s. The magnetic properties were tested with a vibrating sample magnetometer with a 3T electromagnet with 75 Hz, ±5 kOe (+0.5 T).

In vitro CHX Loading Analysis

The percentage of encapsulation-efficiency (EE) and drug-loading (DL) were calculated by determining the amount of free non-encapsulated CHX via Ultraviolet-Visible (UV-Vis) spectroscopy at 289 nm (17,18) The DL (%) and EE (%) were calculated according to the following equations:

$$CHX loading : \qquad Equation 1$$

$$DL ( \% ) = 100 \times ( \frac{mass of loaded CHX in the particles}{mass of particles} ) ,$$

$$CHX encapsulation - efficiency as : \qquad Equation 2$$

$$EE ( \% ) = 100 \times ( \frac{mass of loaded CHX in the particles}{Total mass of CHX} ) , and$$

$$Quantity of loaded CHX = Total CHX - unloaded quantity of CHX . \qquad Equation 3$$

The particles were also tested via thermogravimetric analysis (TGA) using 10 mg of SiQac@Fe₃O₄@m-SiO₂ and Fe₃O₄@m-SiO₂ heated in alumina crucibles from 30 to 800° C., heat flow rate of 10° C./min, under N₂ atmosphere (19).

In vitro CHX Release Analysis

CHX solutions were measured to generate a standard curve via UV-Vis. CHX @Fe₃O₄@m-SiO₂ and CHX@SiQuac@Fe₃O₄@m-SiO₂ (50 mg each) were suspended in 5 mL of phosphate buffer saline (PBS) at room temperature under stirring (100 RPM). Two different pH values (5.5 and 7.4) of PBS were used to mimic the acidic and neutral environments (17). The samples were centrifuged at 7,000 RCF for 10 min, and fresh PBS replaced the solutions with the same pH values. The removed aliquots were analyzed for CHX content using UV-Vis at 289 nm and high-performance liquid chromatography (HPLC). The cumulative release of CHX was calculated according to the following equation 4):

$$CHX release ( \% ) = 100 \times ( \frac{CHX concentration}{Loading concentration} ) . \qquad Equation 4$$

### Inhibition of Human Matrix Metalloproteinases (MMPs)

For the MMPs inhibition assay, SENSOLYTE Generic MMP Assay Kit Colorimetric was used. The MMP thiopeptide substrate solution was diluted to 0.2 mM with the supplied assay buffer in a 1:50 volume ratio. A 96-well plate was used for the assay using four replicates per group. In each well, 40 μL of MMP-9 (20 ng/well), 10 μL of CHX (1 mg/mL) at serial dilutions, and 50 μL of thiopeptide substrate solution were mixed. The fluorescence intensity was measured using a plate reader (SpectraMax M5, Molecular Devices, Sunnyvale, CA, USA) at Ex/Em-490 nm/540 nm with a kinetic mode every 5 min for 60 min (20). The substrate control group was set as a background, where its absorbance was subtracted from other wells. The percentage of MMP inhibition was calculated according to equation 5:

$$MMP inhibition ( \% ) = ( 1 - \frac{absorbance of test compound group - a b s o r bance of test compound control}{absorbance of the positive control - a b s o r b ance of s ubstrate control} ) . \qquad Equation 5$$

### Cytotoxicity Analysis

Human gingival fibroblasts and dental pulp stem cells were used to analyze the particle's cytotoxicity. Human gingival fibroblasts, fibroblast medium, dental pulp stem cells, cell counting, and LIVE/DEAD imaging kits were sourced from Innoprot, Sciencell Research Laboratories, Lonza, Dojindo, and ThermoFisher Scientific, respectively. Human gingival fibroblasts were cultured in a fibroblast medium supplemented with 2% fetal bovine serum, 1% fibroblast growth supplement, 100 IU/mL penicillin, and 100 IU/mL streptomycin. Dental pulp stem cells were cultured in basal medium supplemented with 2% fetal bovine serum, 2 mM L-glutamine, GA-1000, 100 mM ascorbic acid solution, 100 IU/mL penicillin, and 100 IU/mL streptomycin. Different concentrations of CHX@Fe₃O₄@m-SiO₂ and CHX@SiQuac@Fe₃O₄@m-SiO₂ were dissolved in 4 mL of each medium and incubated for 24 h at 37° C. The cells were seeded at 5000 cell/well in a 96-well plate, incubated overnight, and exposed to CHX@Fe₃O₄@m-SiO₂ and CHX@SiQuac@Fe₃O₄@m-SiO₂ for 24 h. CCK-8 solution (10 μL/well) was added, and the plates were incubated for 2 h at 37° C. The absorbance was measured at 450 nm. The cell viability was normalized against the cell viability of untreated cells. The results were expressed as a percentage of viability. For the live/dead assay, HGF and DPSC were seeded in the wells of a 24-well plate 10,000 cells/well), and the medium was changed to allow the viability to be above 90%. Cells were then exposed to CHX@Fe₃O₄@m-SiO₂ and CHX@SiQuac@Fe₃O₄@m-SiO₂ for 24 h at the ratio of 1:1. Live/Dead Cell imaging kit was used to stain the cells and visualized using a fluorescence microscope.

### Antibacterial Evaluation of the Synthesized Particles

The antibacterial activity of CHX@Fe₃O₄@m-SiO₂ and CHX@SiQuac@Fe₃O₄@m-SiO₂ was analyzed using S. mutans cultures. Streptococcus mutans (UA159) and various laboratory reagents including Phosphate Buffered Saline (PBS), Brain Heart Infusion broth and agar, ethanol, Dimethyl Sulfoxide (DMSO), and MTT were sourced from Quality Biological, Research Products International, and Sigma-Aldrich. S. mutans was cultured for 24 h, and its optical density was adjusted using brain heart infusion (BHI) broth to 0.1 at 600 nm. In a 96-well microtiter plate, the concentrations of 1, 2, 3, 4, and 5 wt. % of the particles were added with equal volumes of the diluted S. mutans in BHI broth. The plate was incubated at 37° C. for 24 h in an anaerobic incubator. The total absorbance was measured at 595 nm. The planktonic cells were removed from the plate, leaving the attached biofilm, and 200 μL of 10% formaldehyde was added to each well for 30 minutes to fix the cells. Next, formaldehyde was removed, and the biofilm cells were washed three times with deionized water. 200 μL of 0.5% crystal violet dye was added to each well, and the cells were stained for 30 min. The wells were rinsed three times with deionized water, and 200 μL of 2-isopropanol was added into each well for 1 h to lyse the cells and extract the crystal violet. After that, the plate was read at 490 nm to measure the biofilm formation. The “Initial CFU” stands for “Initial Colony Forming Units.” This term refers to the number of Colony Forming Units in the control group, which was not exposed to the particles investigated in our study. It serves as a baseline measurement to gauge the initial microbial load before any treatment or exposure occurs.

In the context herein, the percentage reduction of S. mutans biofilm growth was evaluated. To do so, the CFU count after the exposure to particles with the Initial CFU count in the control group was compared. The formula for calculating the percentage reduction is as follows:

$$Antibacterial rates ( R ) = ( \frac{Bacteria in control group - Bacteria in t esting group}{Bacteria in control group} ) \times 100 \% . \qquad Equation 6$$

### Dental Adhesive Formulation

A base resin was formulated with bisphenol-A-glycidyl methacrylate (BisGMA) and 2-hydroxyethyl methacrylate (HEMA) combined at 66.66 wt. % and 33.33 wt. %, respectively, to formulate the dental adhesives. Camphorquinone, Ethyl 4-dimethylaminobenzoate, and Butylated hydroxytoluene were procured from Sigma-Aldrich, and Bisphenol-A-glycidyl methacrylate (BisGMA) and 2-hydroxyethyl methacrylate (HEMA) from Esstech, all used as received without further purification. Camphorquinone, ethyl 4-dimethylamino benzoate, and butylated hydroxytoluene were added at 1 mol %, 1 mol %, and 0.01 wt. %, respectively (21). The multifunctional platform (denoted CHX@SiQuac@Fe₃O₄@m-SiO₂) was composed of iron oxide (Fe₃O₄) microparticles (core), mesoporous silica (SiO₂, shell), and chlorhexidine (CHX) encapsulated by the mesoporous silica. The particles were functionalized by an antibacterial quaternary ammonium silane (SiQuac). The particles were added into the adhesive at 2, 3, 4, and 5 wt. %. An adhesive without the particles was assigned as a control group. The dental adhesives were mixed using a mechanical mixer (DAC 150 SpeedMixer, Flacktek, Landrum, SC, USA) at 2800 RPM for 1 min. Groups are described as follows:
a) Group containing adhesive with no addition of CHX@SiQuac@Fe₃O₄@-SiO₂ , called “Control”,
b) Group containing adhesive with addition of CHX@SiQuac@Fe₃O₄@-SiO₂ at 2 wt. %, called “2%”,
c) Group containing adhesive with addition of CHX@SiQuac@Fe₃O₄@-SiO₂ at 3 wt. %, called “3%”,
d) Group containing adhesive with addition of CHX@SiQuac@Fe₃O₄@-SiO₂ at 4 wt. %, called “4%”, and
e) Group containing adhesive with addition of CHX@SiQuac@Fe₃O₄@-SiO₂ at 5 wt. %, called “5%”.

### Degree of Conversion (DC %)

The uncured samples (n=3) (21) were directly dispensed on the ATR (Golden Gate, Specac, Fort Washington, PA, USA) of FT-IR (Nexus 670, Thermo Scientific, Madison, WI, USA) using a silicon mold with 1 mm heigh to contain the adhesive. The samples were photoactivated using a light-emitting diode with 1000 mW/cm² (VALO Cordless, Ultradent Products, South Jordan, Utah, USA) for 20 s. The spectra were acquired before and after the photoactivation using Opus 6.5 Software and Blackman Harris 3-Term apodization with 4 cm⁻¹ resolution and 4 scans/s. The degree of conversion (DC %) was calculated using the high peaks at 1640 cm⁻¹ and 1610 cm⁻¹. The following equation was employed (21):

$$\begin{matrix}Equation 7 & \\ DC ( \% ) = 100 \times ( \frac{peak height of cured aliphatic C = C / peak height of cured aromatic C = C )}{peak height of uncured aliphatic C = C / peak heigh of uncured aromatic C = C} ) . & ( 1 )\end{matrix}$$

### Contact Angle

The contact angle between the water drops and the polymerized adhesives and the contact angle between the uncured adhesives and the dentin surface was tested. For the first test, the sessile drop method was used to measure the static contact angle of water by dropping 10 mL on the polymerized disc-shaped samples of adhesives (n=3, 8 mm in diameter and 1 mm in thickness) (22). After 2 s from the drop placement, three images were captured using goniometer software (Sessile drop method). For the second test, a flat dentin surface was prepared (22), and an adhesive drop of 10 mL was placed on this surface following the sessile drop method.

### Quantification of Charge Density of the Quaternary Ammonium Groups

Disc-shaped adhesive samples (n=6, 8 mm and thickness of 0.5 mm (23) were prepared, immersed in water, and agitated for 1 h to remove uncured monomers. Fluorescein Sodium Salt, Cetyltrimethyl ammonium chloride (CTMAC), and both forms of Sodium phosphate were sourced from Sigma-Aldrich, Saint Louis, MO, USA.

The samples were immersed in a 48-well plate containing 200 ml of fluorescein sodium salt at 100 mg/mL in deionized water. The plate was shaken at 60 RPM for 10 min in the dark at room temperature. The samples were washed three times with deionized water and transferred to a new 48-well plate that contained 200 ml of 0.1 wt. % cetyltrimethylammonium chloride solution in deionized water supplemented with 10 vol % of 100 mM phosphate buffer at pH 8. The 48-well plate was shaken at 60 rpm for 20 min in the dark at room temperature to desorb complex fluorescein dye. The absorbance value was read at 501 nm using the plate reader to yield the concentration of fluorescein dye. Then, fluorescein concentration was calculated using Brees Law and an extinction coefficient of 77 mM⁻¹ cm⁻¹ by performing the following equation:

$$[ Dye ] = ( {Abs}_{501} ) / ( {e}_{501} \times L ) , \qquad Equation 8$$

where [Dye] is the extracted fluorescein concentration, Abs₅₀₁ is the absorbance value at 501 nm, e₅₀₁ is the extinction coefficient that equals 77 mM⁻¹ cm⁻¹ for fluorescein, and L is the length of polystyrene cuvette (1 cm) traversed by spectrophotometer light. The cationic charge density of adhesive discs was calculated by using the following equation:

$$Charge density = [ Dye ] \times V \times N / A , \qquad Equation 9$$

where V is the extraction solution volume, N is Avogadro's number (6.023×10²³), and A is the sample's surface area. Results are then expressed as the total charge molecules per exposed surface area (24).

### Water Sorption and Solubility

The water sorption (WS) and solubility (SL) were performed according to ISO 4049:2009 (25). Disc-shaped samples of adhesives (n=6, 15 mm diameter, 1 mm thickness) (26) were prepared. The samples were placed in a silica gel desiccator at 37° C. for 24 h and measured till constant mass (m¹) was obtained using an analytical balance (VWRâ A-Series, Radnor, PA, USA). The diameter and thickness of each sample were measured using a digital caliper to calculate the volume (v) in mm³. The samples were immersed in 10 mL of distilled water in sealed vials at 37° C. for seven days. Then, the samples were washed under running water and gently wiped with absorbent paper. They were weighed (m²) and returned to the desiccator for 24 hours. Lastly, the samples were weighed till a constant weight was obtained to record the final mass (m³). The WS and SL were calculated according to the following equations:

$$WS ( mg / {mm}^{3} ) = \frac{m 2 - m 3}{v} , and \qquad Equation 10$$

$$SL ( mg / {mm}^{3} ) = \frac{m 1 - m 3}{v} . \qquad Equation 11$$

### Ultimate Tensile Strength (UTS)

Hourglass-shaped samples were prepared (n=5, 8 mm long, 2 mm wide, 1 mm thick, 1 mm² cross-sectional area) (27) using a metallic mold and kept in distilled water at 37° C. for 24 h. The samples were fixed in metallic jigs using cyanoacrylate resin and tested under tension with a universal testing machine (EZ-SX Series, Shimadzu, Kyoto, Japan) at 1 mm/min crosshead speed. The samples were fractured at the constricted area; the values were acquired in newton (N) and divided by the constriction area (mm²) to be expressed in megapascal (MPa).

Micro Shear Bond Strength (m-SBS)

Extracted human third molars (n=12) were used after the approval of the institutional board of this University (HP-00088564). The roots were separated at the cementoenamel junction under running water, the crowns were individually embedded in self-curing acrylic resin, and the dentin was exposed and flattened with a silicon carbide sandpaper (600-grit) for 30 s with distilled water irrigation. The samples were rinsed for over 1 min, dried, and the dentin was etched (37% phosphoric acid for 15 s), rinsed for 30 s, and gently dried using absorbent paper. A primer (Scotch Bond Multipurpose, 3M EPSE, Monrovia, Calif., USA) was applied for 20 s using a micro brush and air-dried. The dental adhesives were applied with a micro brush and photoactivated for 20 seconds. A commercial resin composite (TPH Spectra ST LV, Dentsply Sirona, York, PA, USA) was used to fill polyethylene tubes placed on the adhesive-treated dentin surfaces with an adhesive area of 0.44 mm² (TYGON Medical Tubing Formulations 54-HL, Saint Gobiain Performance Plastics, Akron, OH, USA) and covered by Mylar strips. The resin composites were photoactivated for 20 s on the top, and the samples were stored in distilled water for 24 h at 37° C. The samples were tested in a computer-controlled Universal Testing system (Insight 1, MTS, Eden Prairie, MN, USA) at 1 mm/min. The maximum force in Newtons (N) was obtained for each sample and then divided by the cross-sectional area of the resin composite cylinders to yield the data in megapascals (MPa) (28).

Microtensile Bond Strength (μ-TBS) under Magnetic Field in a Simulated Pulpal Pressure Model

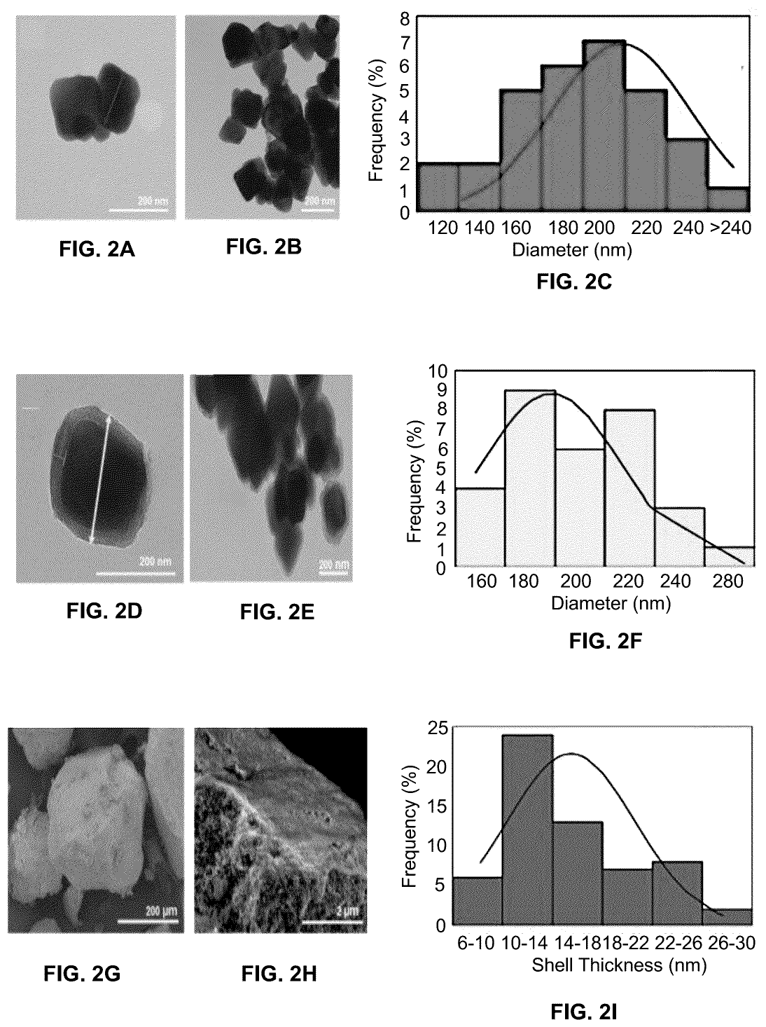

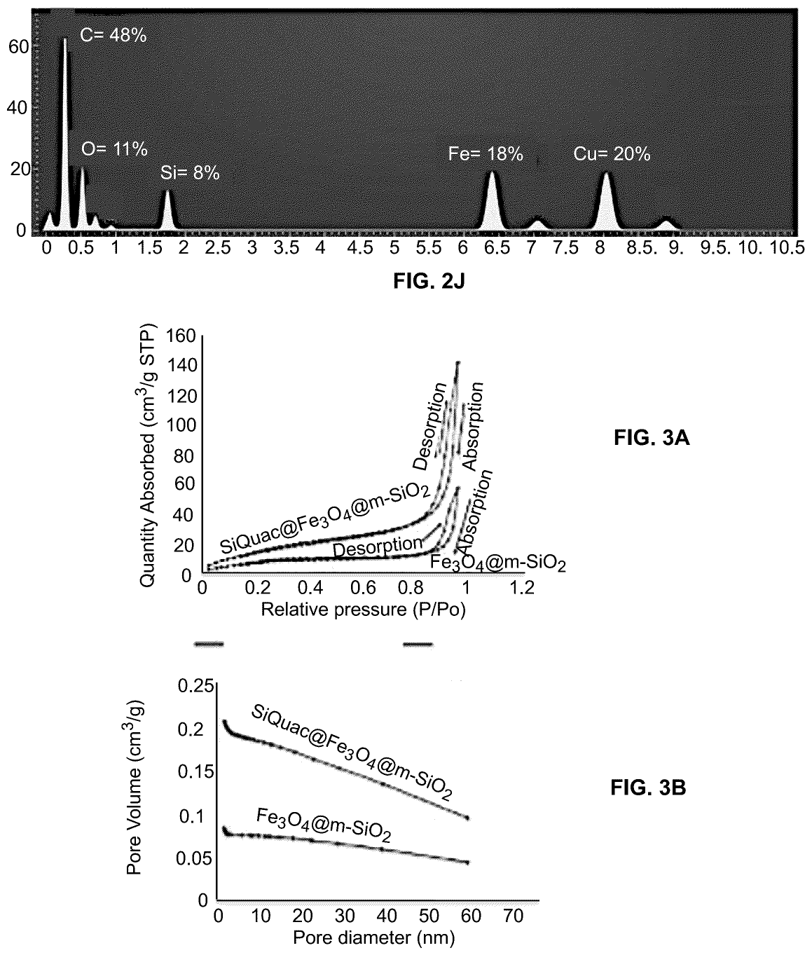

Extracted human third molars (n=5) were used after the approval of the institutional board of this University (HP-00088564). The teeth were sectioned transversally with a slow-speed diamond saw (Isomet, Buehler, Lake Bluff, IL, USA) in two areas: 1) to expose the dentin and 2) to remove the roots (FIG. 2C). The pulpal tissue was removed, and simulated pulp pressure was applied under a magnetic field (FIG. 2C) (1, 28).

For the magnetic field, a Neodymium magnet (NdFeB, NIB, or Neo magnet; grade N42 coated with Nickle-Copper-Nickel triple-layer, size 1.5″×1.5″) was fixed 12 mm away from the flat mid-coronal dentin using a custom 3D support (Polylactic acid-PLA filament, melted Extrusion Modeling, Creality Ender 3 Pro Printer).

The groups with the best performance outcomes in the μ-SBS data were tested for microtensile bond strength (μ-TBS) in addition to the control group at two different time points: baseline and aged. The dentin was treated according to the previously described μ-SBS for the bonding procedures. Two 2-mm increments of the commercial resin composite were placed on dentin and photoactivated for 20 seconds each. The samples were stored in distilled water for 24 h at 37° C. and sectioned into beams of 0.7×0.7 mm under distilled water (Isomet; #15HC blade 127×0.04 mm). The beams were fixed in metallic jigs using cyanoacrylate resin and tested under tension using the universal testing machine (Insight 1, MTS, Eden Prairie, MN, USA) at 1 mm/min until fracture. The values of beams fractured at the interface were recorded in N, divided by the bonding area, and expressed in MPa (28).

The failure pattern was analyzed and classified as adhesive (failure along the adhesive interface), cohesive in dentin (failure within dentin substrate), and cohesive in composite (failure within the resin composite) (29). Half of the beams per tooth were tested immediately after 24 hours from the restoration placement, and another half was tested after thermocycling aging (Odeme Thermocycling, OMC-250 L, Luzerna, Brazil) with computer-controlled water baths of 5 and 55° C. to simulate six months of clinical service. Each cycle consisted of 30 seconds of immersion (dwell time) and 5 seconds of transfer time.

Sample Preparation for the S. mutans Biofilm Model

Disc-shaped adhesive samples (n=6, 8 mm diameter, and 1 mm thickness) were prepared and stored for 24 h at 37° C. Samples were sterilized using ethylene oxide (Anprolene AN 74i, Anderson Products, Haw River, NC, USA) for 24 hours and degassed for seven days. S. mutans UA159 was used in the biofilm mode (30) after being overnight cultured for 18 h at 5% CO₂ and 37° C. in brain heart infusion (BHI) broth. After that, the culture was supplemented by 1 wt. % of sucrose and added to BHI broth to prepare the inoculum. The adhesive samples were placed at the bottom of a 24-well plate, the inoculum was added to each well, and the plate was incubated for 24 hours at 5% CO₂ and 37° C. After 24 hours, the medium was changed by a fresh BHI broth supplemented with 1 wt. % of sucrose and incubated for 24 hours to develop a 48-hour biofilm.

### Colony Forming Unit (CFU) Counting Assay

Each biofilm sample was placed in 1 mL of cysteine peptide water (CPW) in a glass vial to detach the biofilm by 10 s vortex, 1 m sonication (CGOLDENWALL Ultrasonic Homogenizer, SPW Industrial, Laguna Hills, CS, USA), and vortex again for 10 s. The bacterial suspension of each sample was serially diluted, and three drops of 10 μL from each dilution were plated onto BHI agar plates and incubated for 48 h at 5% CO₂ and 37° C. The number of colonies was visually counted with a loupe (Reichert Quebec Darkfield Colony Counter, Depew, NY, USA), and the CFU/mL was calculated according to the dilution factor.

### Metabolic Activity Assay

The biofilm samples grown over the tested adhesives (n=6) were transferred to a new 24-well plate containing 1 mL of tetrazolium dye and incubated for 1 h at 37° C. in 5% CO₂. The samples were transferred to a new 24-well plate containing 1 ml of dimethyl sulfoxide for 20 min at room temperature in the dark to dissolve the formazan crystals. The solution from each well (200 μL) was transferred to a 96-well plate to measure the absorbance at 540 nm using a plate reader (31).

### Fluorescence Microscopy Staining

The biofilm samples grown over the tested adhesives (n=1) were rinsed with phosphate-buffered silane to remove non-adherent bacteria. Live/dead BacLight kit (Molecular Probes Eugene, OR) was used to stain samples by applying a mixture of 2.5 mM of each SYTO 9 and Propidium iodide for 15 min. Dead bacteria were stained by propidium iodide, emitting a red fluorescence, while live bacteria were stained by SYTO 9, emitting a green fluorescence. Samples were imaged at five random locations using an inverted fluorescence microscope (Eclipse TE2000-S, Nikon, Melville, NY).

### Scanning Electron Microscopy of Biofilm Grown Over the Material

Images were obtained to evaluate the load and morphology of S. mutans biofilm formed in 48 h on adhesive samples. Samples were washed with PBS to remove unattached biofilm, then fixed using 3% formaldehyde overnight. Biofilm was then dried using ethanol dilutions followed by 100% hexamethyldisilazane. Samples were sputter-coated with platinum; images were then captured using SEM at magnifications of X500, X3,000, and X8,000 with 3.5 kV (Quanta 200, FEI Company, Hillsboro, OR, USA).

### Long-Term Antibacterial Properties

Samples (n=6) were aged by thermocycling (Odeme Thermocycling, OMC-250 L, Luzerna, Brazil) with computer-controlled water baths of 5 and 55° C. and subjected 15,000 thermocycles to estimate an 18-month clinical service (32-34). Then, samples were sterilized as described. Antibacterial tests were performed as described, and the results were recorded as aged data.

### Cell Culture

The cytotoxicity was assessed using human gingival fibroblast (HGF) and dental pulp stem cells (DPSC). HGF (passage 7) was cultured in a fibroblast medium supplemented with 2 wt. % fetal bovine serum, 1 wt. % fibroblast growth supplement, 100 IU/mL penicillin, and 100 IU/mL streptomycin. DPSC (passage 6) were cultured in DPSC basal medium supplemented with 2 wt. % fetal bovine serum, 2 mM L-glutamine, GA-1000, 100 mM ascorbic acid solution, 100 IU/mL penicillin, and 100 IU/mL streptomycin.

### Cell Viability

Cells were seeded into a 96-well plate at 5,000 cells/well density in the corresponding medium. The disc-shaped adhesive samples (n=3, 4 mm diameter, and 1 mm thick) were prepared and sterilized as described for the antibacterial analyses. Each sample was immersed in a 4 mL medium for 24 h at 37° C. This procedure results in a surface area/solution ratio of 0.63 cm²/mL, following the recommendation of ISO 10993-12:2021 (0.5-6 cm²/mL range). Original extracts were diluted with fresh medium at 2-,4-,8-,16-,32-,64-, and 128-fold. The concentrations expressed in weight percentage (1 to 5 Wt. %), correspond to 10,000 to 50,000 mg/L. Then, 100 mL of each sample's original and diluted extracts were added to the cultured cells and incubated for 24 h at 37° C. with 5% CO₂. After that, 10 mL of cell counting kit-8 (CCK-8) (WST®-8, Selleckchem, Houston, TX, USA) was added to each well and incubated for 2 h at the same previous conditions. Absorbance was measured at 450 nm to represent the cellular dehydrogenase activity of live cells in culture media, with the control group consisting of cells grown in the media alone.

### Cell Imaging

In a 24-well plate, cells were seeded at 40,000 cells/well. Original and diluted extracts were added to the cultured cells and incubated for 24 h at 37° C. with 5% CO₂. Live/Dead Cell Imaging Kit was used to stain the cells for 15 minutes, and then cells were visualized using an inverted epifluorescence microscope (Eclipse TE2000-S, Nikon, Melville, NY, USA).

### Statistical Analysis

All biological experiments were performed in triplicate; the average of the three experiments per sample was considered a statistical unit and expressed as means and standard deviation. Data normality was evaluated using the Shapiro-Wilk test SPSS, version 28.0 (SPSS Inc., Chicago, IL, USA). Experiments were assessed for significance using a One-way Analysis of Variance (ANOVA) followed by Tukey's post hoc test. Immediate and long-term comparisons were performed using paired t-tests within each group. A level of significance of 5% was applied.

### EXAMPLE 2

### Results

### Physical Characterization of the Synthesized Particles

TEM characterization evidenced the cubic Fe₃O₄ particles (mean size of 191±30 nm) with a non-normal distribution size (FIGS. 2A, 2C). Fe₃O₄@m-SiO₂ TEM images showed an irregular morphology of cubic and spherical particles with a uniform thin shell (FIG. 2D). Less aggregation was noticeable in Fe₃O₄@m-SiO₂ particles than in Fe₃O₄ particles (FIGS. 2B, 2E). The silica coating amplifies the chemical stability of the magnetic particles, particularly when exposed to acidic environments, hindering their tendency to agglomerate. The uniform, thin silica shell enhances chemical stability, as reported by Trzeciak et al. (35), and facilitates the formation of stable molecular attachments, critical for drug delivery applications in dentistry.

The mean particle size for Fe₃O₄@m-SiO₂ was 172±41 nm, while the shell thickness was 15.4±5 nm (FIGS. 2F, 2I). TEM and SEM images of CHX@SiQuac@Fe₃O₄@m-SiO₂ showed that the nanoparticles are cubical rhomboidal with a semi-smooth surface, confirming the irregular particle shape resulting from the complex decomposition process (FIGS. 2G, 2H). The observed cubic shapes of Fe₃O₄ and Fe₃O₄@m-SiO₂, coupled with the reduction in aggregation due to the silica coating, align with findings by Sajid et al. (36), who highlighted the importance of particle shape and aggregation in influencing the mechanical properties of dental composites.

A significant finding from the EDS results is the approximately 46 wt. % carbon doped in the mesoporous silica. This high carbon content is indicative of the presence of chlorhexidine (CHX) (FIG. 2J). The EDS results also suggest the potential for further functionalization of these particles. The silanol groups on the surface of the silica coating, as evidenced by the presence of Si, serves as reactive sites for bioconjugation (37). The silanol groups on the surface of the silica coating serve as reactive sites, facilitating the formation of stable and tailored molecular attachments, expanding the magnetic particles' functionality for healthcare applications (38).

### Surface Analysis & Saturation Magnetization

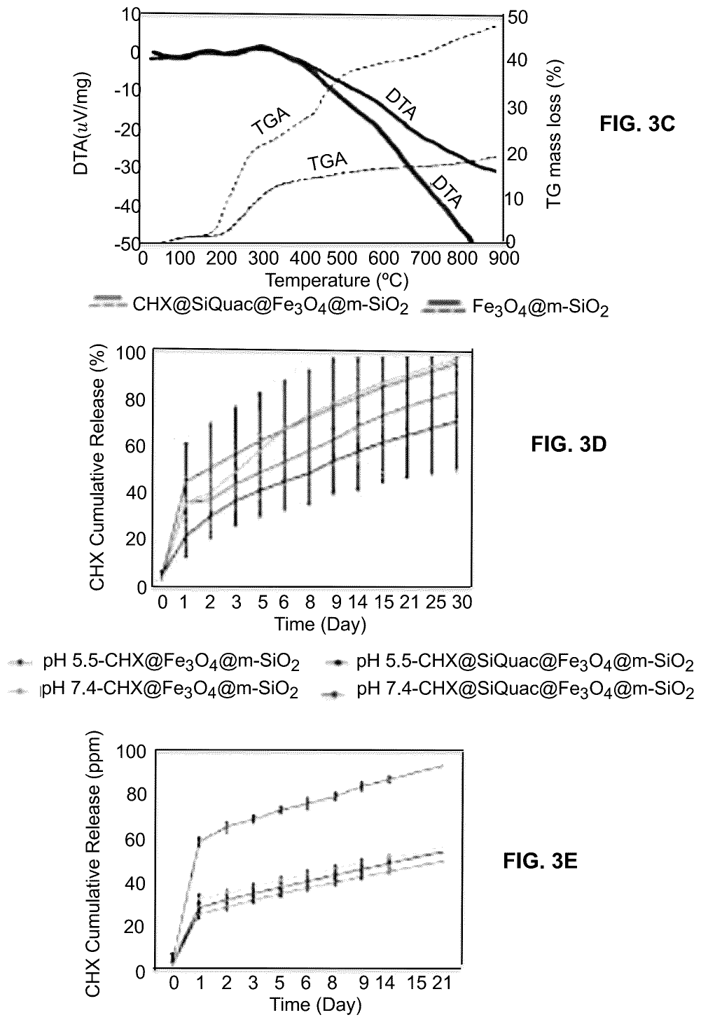

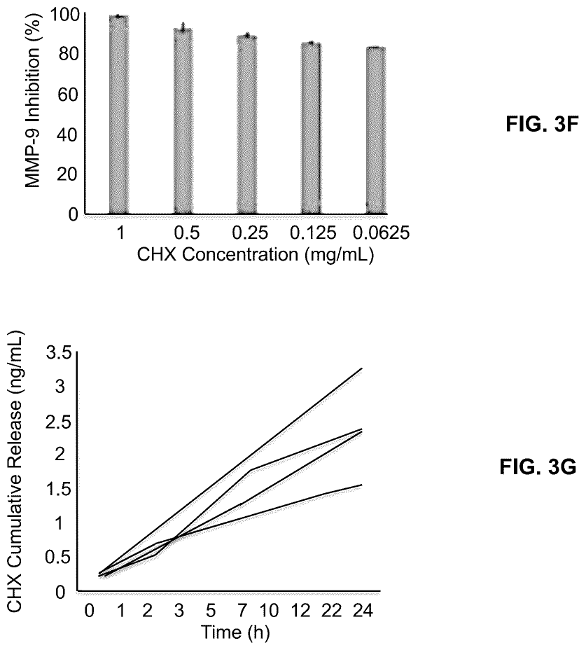

FIGS. 3A-3J provides a comprehensive characterization of the materials developed, including surface area, pore size, X-ray diffraction, saturation magnetization, and the short-term (initial 24 hours) and long-term (21 and 30 days) chlorhexidine (CHX) release profile. Additionally, it illustrates the impact of chlorhexidine on MMP-9 and presents a schematic of the loading process.

In FIGS. 3A-3B, Fe₃O₄@m-SiO₂ and SiQac@Fe₃O₄@m-SiO₂ displayed a type IV isotherm, indicative of their mesoporous nature (39). The H1 hysteresis loop in SiQac@Fe₃O₄@m-SiO₂, particularly prominent at 0.8<P/Po<1.0, suggests a larger pore volume than Fe₃O₄@m-SiO₂. The mesoporous size distribution peaked at 1.8 nm for Fe₃O₄@m-SiO₂ and 1.9 nm for SiQac@Fe₃O₄@m-SiO₂, fitting within the IUPAC classification for mesopores (40). The surface area and pore volume of Fe₃O₄@m-SiO₂ were 37.7 m²/g and 0.085 m³/g, respectively. At the same time, for SiQac@Fe₃O₄@m-SiO₂, these values were 66.7 m³/g and 0.21 m³/g, respectively. These findings support an expanded surface area, facilitating a more efficient drug loading and a controlled release mechanism. This attribute is highly beneficial.

An X-ray diffraction of both Fe₃O₄@m-SiO₂ and CHX@SiQuac@Fe₃O₄@m-SiO₂ exhibited characteristic peaks of a face-centered cubic system (41), typical of magnetic Fe₃O₄ with a spinel structure (42). The diffraction peaks corresponded to various crystallographic planes, with a dominant crystalline phase in Fe₃O₄@m-SiO₂ and a more amorphous phase in CHX@SiQuac@Fe₃O₄@m-SiO₂ due to SiQuac and CHX.

A M-H (magnetization=M and field strength=H) magnetic hysteresis loop of CHX@SiQuac@Fe₃O₄@m-SiO₂ at room temperature displayed symmetric hysteresis and saturation magnetization, indicating ferrimagnetic properties. Results (not shown) showed symmetric hysteresis and a saturation magnetization of 61.3 emu/g, similar to 53-57 emu/g values as Balhaddad et al. (43) reported, using SPIONs. The superparamagnetic property, characterized by the absence of remanence or coercivity, suggests that the particles respond to external magnetic fields without retaining residual magnetism once the field is removed. This feature is crucial for potential controlled movement within dental resins, as it minimizes the risk of unwanted magnetic interactions post-treatment.

A characteristic absorption peak of the Fe₃O₄ core at 579 cm⁻¹ related to Fe—O vibration (44), and 1085 cm⁻¹ associated with Si—O—Si vibrational band was obtained by FTIR analysis. In CHX@SiQuac@Fe₃O₄@m-SiO₂ spectra, the presence of SiQuac is confirmed by the characteristic vibrations of quaternary amine NR₄+ at 950 cm⁻¹. In contrast, the occurrence of multiple vibrations evidences the existence of CHX at 1093, 2947, and 3325 cm⁻¹ associated with the bands C—N, C—H, and N—H, respectively (45). FIG. 3C shows a thermogravimetric analysis (TGA) where the weight loss stages were used to assess the degree of organic functionalization on both CHX@SiQuac@Fe₃O₄@m-SiO₂ and Fe₃O₄@SiO₂. The TGA findings indicated no significant weight loss occurred below 200° C. for CHX@SiQuac@Fe₃O₄@m-SiO₂ and Fe₃O₄@SiO₂. This observation suggests excellent thermal stability of the base materials up to this temperature, which is essential for their use in dental applications where materials may be subjected to varying temperatures during processing and use.

The weight loss observed between 200-800° C. can be primarily attributed to the quantity of organic material attached to the nanoparticles. Notably, the weight loss in CHX@SiQuac@Fe₃O₄@m-SiO₂ was higher than in Fe₃O₄@SiO₂, indicating greater organic functionalization. This is a significant finding as it confirms the successful attachment of CHX and SiQuac to the Fe₃O₄@m-SiO₂, which is a key factor in the antibacterial and drug-release properties of the nanoparticles. A study by Azadpour et al. (46) reported a similar trend in the thermal degradation of functionalized magnetic nanoparticles but with a slightly lower onset of degradation, suggesting a difference in the thermal stability of their functional groups.

In vitro CHX Loading and Release Profile

Differential thermal analysis (DTA) was utilized to determine the loading quantity of chlorhexidine (CHX) into the synthesized Fe₃O₄@m-SiO₂. An initial weight loss (1.5 wt. %) was observed under 100° C., which can be attributed to the evaporation of physically absorbed solvents. There was almost no mass loss between 100° C. and 165° C. in the analysis of Fe₃O₄@m-SiO₂ and between 100° C. and 200° C. in CHX@SiQuac@Fe₃O₄@m-SiO₂. DTA curves showed a weak and broad peak at 140° C. in Fe₃O₄@m-SiO₂ and 186° C. in CHX@SiQuac@Fe₃O₄@m-SiO₂. CHX loading efficiency was 68%+3.7% (FIG. 3C) indicating a high capacity for drug incorporation.

For further analysis, CHX cumulative release was evaluated using UV-vis spectroscopy (FIG. 3D) and high-performance liquid chromatography (HPLC) (FIG. 3E). CHX@Fe₃O₄@m-SiO₂ and CHX@SiQuac@Fe₃O₄@m-SiO₂ demonstrated an increased CHX cumulative release across the tested pH levels over time. Notably, in FIG. 3E, the presence of SiQuac significantly enhanced the release of CHX at both pH 5.5 and pH 7.4 compared to the CHX-only condition.

The rationale for employing UV-Vis spectroscopy and HPLC lies in their complementary strengths for analyzing drug release. UV-Vis spectroscopy is a widely used method for its rapidity and ease of use, providing a quick and reliable way to monitor the concentration of CHX in solution. On the other hand, HPLC offers a higher specificity and accuracy level. It can separate CHX from other components in the sample, thus providing a more precise measurement of its concentration. This is crucial in complex systems where multiple components might interfere with the measure. HPLC is especially valuable for confirming the release kinetics of CHX and ensuring the accuracy of the release data (47).

The selection of pH 7.4 and 5.0 for the release study reflects the physiological and cariogenic conditions of the oral cavity. pH 7.4 simulates the typical saliva environment, while pH 5.0 mimics the acidic conditions often related to tooth decay development. The enhanced release of CHX in the presence of SiQuac at both pH levels is a significant finding, indicating the material's versatility and effectiveness across a range of oral environments.

Interestingly, demonstrating efficient CHX release at pH 7.4, challenge the notion that drug release is typically more efficient at lower pH levels. This phenomenon is thought to be influenced by the presence of SiQuac, a quaternary ammonium compound, which typically shows resistance to acidic conditions. In neutral to alkaline conditions, where SiQuac is more stable, the release of CHX might be slower and more controlled. In contrast, under acidic conditions (lower pH), the stability of SiQuac could be slightly compromised, potentially leading to a different release profile.

### MMPs Inhibition by CHX

Chlorhexidine, known for its effectiveness as a matrix metalloproteinase inhibitor, shows varying degrees of inhibition efficacy, ranging from partial to complete, particularly at concentrations between 0.2% and 2% (48). MMP-9 and MMP-2 are prevalent in dentin. The impact of CHX on MMP-9 was assessed using concentrations of 1, 0.5, 0.25, 0.125, and 0.0625 mg/mL (FIG. 3F). There was a direct correlation between CHX concentration and its inhibitory effectiveness. Specifically, at a 1 mg/mL concentration, CHX achieved a remarkable 99.8% inhibition of MMP-9. This finding indicates that CHX concentrations greater than 1 mg/mL could effectively inhibit MMP-9.

Complementing these results, FIG. 3G showcases the release profile of CHX from the tested formulations, both with (CHX@SiQuac@Fe₃O₄@m-SiO₂) and without silane (CHX@Fe₃O₄@m-SiO₂), under both neutral and acidic conditions over 24 hours. Notably, the released CHX concentrations exceeded 1 mg/mL, suggesting that the CHX@SiQuac@Fe₃O₄@m-SiO₂ formulation can release CHX at concentrations effective for MMP-9 inhibition. Thus, the present invention demonstrates the ability of CHX released from CHX@SiQuac@Fe₃O₄@m-SiO₂ to inhibit MMPs in dental applications.

Cytotoxicity Analysis of CHX@SiQuac@Fe₃O₄@m-SiO₂

The culture extract testing method for cytotoxicity screening was used as an MTT assay, focusing on two cell types clinically relevant to the intended application (FIG. 4). The choice of DPSC and HGF cells for cytotoxicity screening is directly tied to the clinical context of dental adhesive applications. In cavity preparations, especially in deep dentin close to the pulp, DPSCs are critical. Dental adhesives used in such areas must ensure compatibility and non-toxicity to preserve the vitality of the pulp tissue and its regenerative potential. Conversely, HGF cells represent the gingival tissue that interfaces with the margins of bonded restorations.

Light microscopy was employed to observe the cell growth of dental pulp stem cells (DPSC) in contact with CHX@SiQuac@Fe₃O₄@m-SiO₂, as shown in FIGS. 4A-4C. A live-dead fluorescence imaging technique was also used to illustrate cell viability (FIG. 4D). Extracts for testing were prepared by immersing various concentrations (0 wt. %=control), 1, 2, 3, 4, and 5 wt. %) of CHX@Fe₃O₄@m-SiO₂ and CHX@SiQuac@Fe₃O₄@m-SiO₂ in culture mediums at 37° C. for 24 hours. Notably, DPSCs exhibited a significant increase in cell viability at concentrations of CHX@Fe₃O₄@m-SiO₂ above 3 wt. %. A similar trend was observed with the presence of SiQuac, where the viability was comparable to the control at similar concentrations. All tested concentrations fell within the ISO 10993-5 threshold for cell viability (FIG. 4E).

When human gingival fibroblasts were exposed to the particles, the viability pattern mirrored that of DPSC cells, with statistically significant differences only observed at concentrations above 3%. Yet, all remained within the acceptable viability threshold (FIG. 4F). FIGS. 4G-I further illustrate the growth of HGF cells in contact with CHX@SiQuac@Fe₃O₄@m-SiO₂.

The non-cytotoxic nature of CHX@Fe₃O₄@m-SiO₂ and CHX@SiQuac@Fe₃O₄@m-SiO₂, as demonstrated by their acceptance within the ISO 10993-5 standard, is indicative of their safety for clinical use. Reports of the cell viability of Fe₃O₄ (49), CHX (50), and SiQuac (51) have also shown a concentration-dependent profile, and results were within the ISO 10993-5 recommendations with viability higher than 70% (52). Their ability to enhance cell viability in both DPSC and HGF cells, especially at higher concentrations without inducing cytotoxicity, highlights their use as biocompatible materials suitable for use in areas close to the pulp and gingiva.

Antibacterial Assessment of CHX@SiQuac@Fe₃O₄@m-SiO₂ Particles

The antibacterial assessment of the CHX@SiQuac@Fe₃O₄@m-SiO₂ was conducted against Streptococcus mutans, a key pathogen in dental caries development, in two distinct stages of complexity: planktonic and biofilm stages. This dual-stage testing is crucial for evaluating the effectiveness of new formulations against the different forms of bacterial growth. Planktonic bacteria represent the free-floating individual bacterial cells, while biofilm represents a more complex, community-based stage where bacteria are embedded in a protective matrix. The ability of CHX@SiQuac@Fe₃O₄@m-SiO₂ particles to combat S. mutans in both these stages is relevant for its efficacy in preventing dental caries. The isolated antibacterial performance of the particles was evaluated to illustrate their use as additives for dental adhesive formulations.

FIGS. 4K-4L illustrate the percentage reduction of S. mutans growth in planktonic and biofilm stages, respectively, after exposure to CHX@Fe₃O₄@m-SiO₂ and CHX@SiQuac@Fe₃O₄@m-SiO₂ particles. Both particles demonstrated a concentration-dependent increase in bacterial reduction. Specifically, a 1 wt. % concentration of CHX@Fe₃O₄@m-SiO₂ and CHX@SiQuac@Fe₃O₄@m-SiO₂ significantly reduced planktonic bacteria.

Regarding biofilm reduction, CHX@Fe₃O₄@m-SiO₂ particles required concentrations above 3 wt. % to reduce the biofilm significantly by at least 60%. In contrast, CHX@SiQuac@Fe₃O₄@m-SiO₂ particles, which include antibacterial silane, were more effective, achieving at least a 78% reduction in biofilm at concentrations over 3 wt. % (FIG. 4L).

The distinction between the antibacterial effects on planktonic and biofilm stages of S. mutans is decisive. The dental plaque that grows over the teeth and dental bonded restorations is mainly presented in a biofilm stage (53). Biofilms are inherently more resistant to antibacterial agents due to their protective matrix and the communal nature of bacterial cells (54). This increased resistance explains why lower concentrations (1 wt. % and 2 wt. %) of the dental adhesives were effective against planktonic cultures but less against biofilms. The higher concentration threshold for significant biofilm reduction highlights the challenge in developing effective dental materials against more resilient bacterial communities.

The antibacterial activity of CHX@SiQuac@Fe₃O₄@m-SiO₂ particles is mainly attributed to the release of CHX and the positive charge density of the quaternary ammonium segment in the SiQuac structure without the release of these methacrylates derived from quaternary ammonium salts (13, 22, 55).

### Dental Adhesives Physicochemical Properties

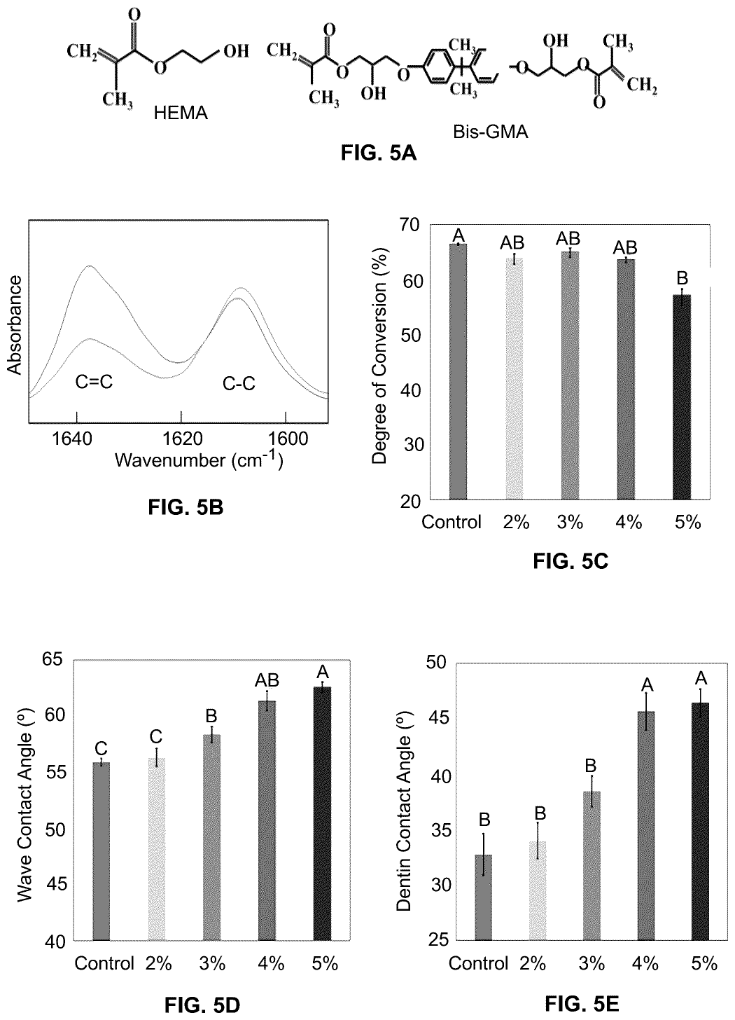

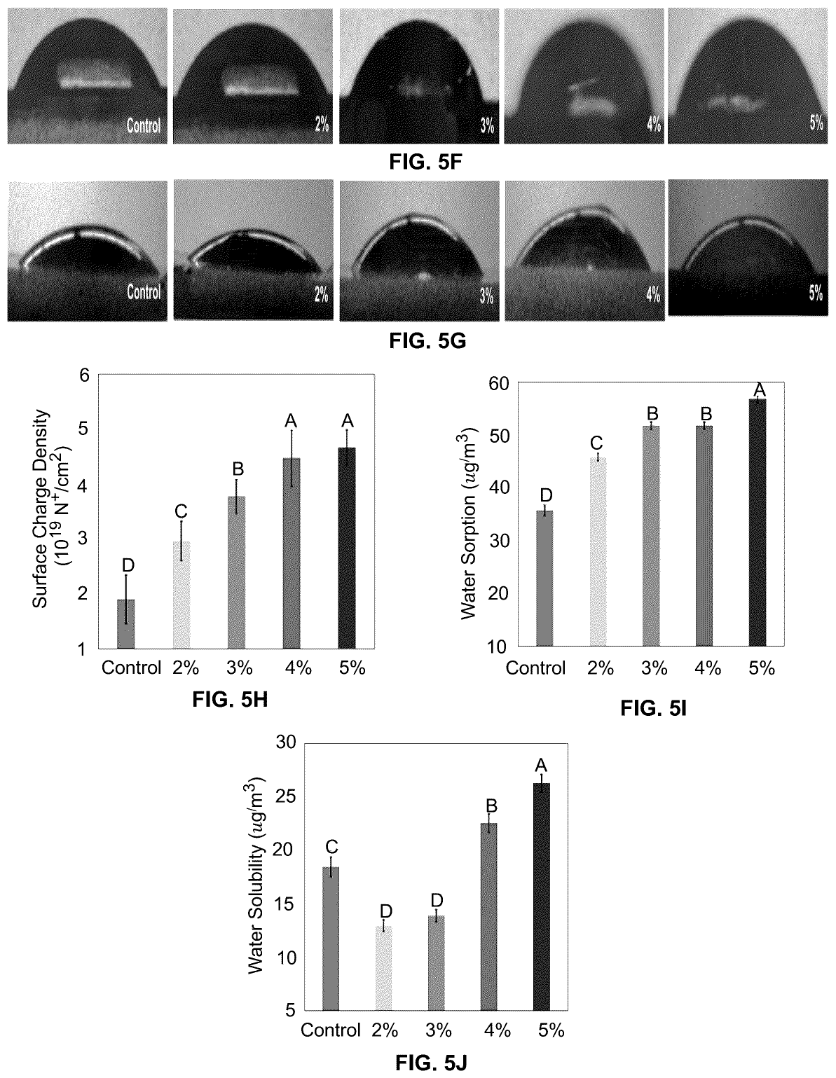

FIGS. 5A-5J present a comprehensive analysis of the physicochemical properties of dental adhesives with different concentrations of CHX@Fe₃O₄@m-SiO₂ and CHX@SiQuac@Fe₃O₄@m-SiO₂, with the results suggesting that higher concentrations may improve certain aspects of adhesive performance, such as conversion degree and wettability, but could also increase water sorption and solubility. The molecular structures of the leading dental adhesive monomers, Hydroxyethyl methacrylate (HEMA) and Bisphenol A-glycidyl methacrylate (Bis-GMA), highlighting the carbon-carbon double bonds (C═C) in red and the Infrared (IR) spectra displaying the peaks for carbon-carbon double bonds (C═C) and single bonds (C—C), which are indicative of the polymerization status are illustrated in FIGS. 5A-5B.

FIG. 5C represents a bar graph showing the degree of conversion (%) for dental adhesives with varying concentrations of CHX@Fe₃O₄@m-SiO₂ and CHX@SiQuac@Fe₃O₄@m-SiO₂ from 2 to 5 wt. %, in comparison to an untreated control, reflecting the extent of monomer-to-polymer transformation.

The initial chemical property assessed in the adhesive formulations was the degree of conversion (DC %). This parameter is critical as it directly impacts the adhesives' mechanical robustness and biological compatibility (56). Achieving a high DC % is vital for optimal performance; a suboptimal DC % can have several negative consequences (57). Firstly, it may release unreacted monomers, potentially compromising the adhesive's structural integrity. Secondly, these unconverted monomers can elevate cytotoxicity, posing a risk to the surrounding biological tissue. Thirdly, residual monomeric components have been shown to influence biofilm development through gene expression regulation, potentially exacerbating bacterial proliferation on the adhesive surface (58). Thus, ensuring a high DC % is integral to dental adhesive applications' overall efficacy and safety.

The adhesives formulated as a base resin herein were modeled after a gold-standard, commercial three-step etch-and-rinse adhesive consistently exhibited high conversion (DC %) across all tested particle concentrations. In the evaluation of particle incorporation into the dental resin, concentrations up to 4 wt. % showed no statistically significant difference in the degree of conversion (DC %) when compared to the control (p<0.05). However, incorporating CHX@SiQuac@Fe₃O₄@m-SiO₂ at 5 wt. % resulted in a statistically significant decrease in DC % (p>0.05). The observed reduction in the degree of conversion at higher concentrations is likely attributable to impaired light penetration through the resin matrix.

The first chemical property analyzed for the adhesives was the degree of conversion (DC %). The achievement of a proper DC % is crucial because it affects the mechanical and biological properties of adhesives (3). A low DC % can lead to 1) the release of unconverted monomers, 2) an increase in cytotoxicity, and 3) the influence of biofilm development via gene regulation since monomer segments can increase bacterial growth (59).

The static contact angle measurement is commonly used to study surface wettability, adhesion, and spreading (60). A contact angle less than 90° indicates a wetting surface, a larger than 90° points to a non-wetting surface, and a zero-contact angle indicates complete wetting (60). The higher the particle concentration, the higher the observed contact angle, as shown in FIGS. 5D and 5F. The groups with 4 and 5 wt. % significantly increased by around 6° (±0.8) compared to the control group (p<0.05). There was no statistical difference between the control and group with 2 wt. % of particles (p>0.05). The mean angle recorded for Group 2% was 56.3 (±0.8), and this value increased to 62.56 (±0.4) for Group 5% (p<0.05). The contact angle of all groups was less than 90°, which indicates that all samples are hydrophilic. Still, their hydrophilicity decreases as the nanoparticles' concentrations increase, probably because of SiQuac's presence (60). The same pattern was observed on dentin samples with adhesive droplets on their surface (FIGS. 5E and 5G). The adhesives with 4 and 5% on dentin significantly increased the contact angle compared to the other groups (p<0.05). A higher particle concentration affects the degree of conversion and hydrophobicity of the adhesive. In discussing the interplay between physicochemical properties and the performance of dental adhesives, the present invention indicates that enhancements in specific properties may necessitate compromises in others. This balance underscores the importance of fine-tuning the adhesive formulations and assessing the concentrations that can maximize benefits while mitigating any adverse effects on the material's core characteristics (61). Achieving an optimal formulation requires carefully considering how adjustments to the adhesive's composition influence its overall function and reliability in clinical scenarios.

FIG. 5H demonstrates the surface charge density values of the formulated dental adhesives. Overall, increased charge density was associated with the increased concentration of CHX@SiQuac@Fe₃O₄@m-SiO₂ in dental adhesives. Compared to the control, Group 5% showed around a 60% increase (p<0.05). The positively charged ammonium group (N⁺) promoted by SiQuac on the sample's surface can attract the negatively charged bacterial cell membrane, which disturbs the cell membrane and leads to a cytoplasmic leakage (62). A previous study reported that the higher the surface density, the more killed bacterial cells (63). At the same time, another study suggested that a positive charge density charge density higher than 10¹⁵ N+/cm² is critical to induce an antibacterial effect (64). Herein, all investigated adhesive groups revealed a charge density greater than 10¹⁵ N+/cm².

In addition, these results can correlate to the amount of CHX released from the surface.

For water sorption (WS) and solubility (SL), significant differences were noted across the groups (p<0.05), as shown in FIGS. 5I-5J. Specifically, water sorption exhibited a 37.2% increase with a rise in nanoparticle concentration relative to the control. Meanwhile, solubility was 29.7% higher in the 5% group when compared to the control. The 2% and 3% groups displayed significantly lower solubility than the control (p<0.05). The chemical composition of the prepared adhesives (i.e., hydrophobic and hydrophilic monomers and filler particles) can directly affect their degradation profile. Dental adhesives' monomers contain hydroxyl, carboxyl, and hydrophilic ester groups, making the formulated adhesive more prone to water sorption (65). High water sorption increases the plasticizing effect, decreasing the mechanical properties (66).

While the contact angle tests indicated an increase in surface hydrophobicity, prolonged water immersion, as simulated in the water sorption and solubility tests, revealed that higher concentrations of particles corresponded to increased water uptake and solubility. The authors suggest that water may permeate the microvoids within the material over time, and the interaction between chlorhexidine and the hydroxyl groups on the silica surface might play a more pivotal role than the initial hydrophobicity observed. This dynamic is thought to significantly influence the results seen in the water sorption and solubility tests.

Moreover, it was observed that despite the higher water sorption and solubility values in the groups with elevated particle concentration, the long-term micro-tensile bond strength (μ-TBS) for the 4% and 5% groups showed an improvement compared to the control. This suggests that the initial increase in hydrophobicity and the interactions between CHX and silica may contribute positively to the adhesive's bonding performance over time despite the higher propensity for water uptake.

### Dental Adhesives Mechanical and Bonding Performance

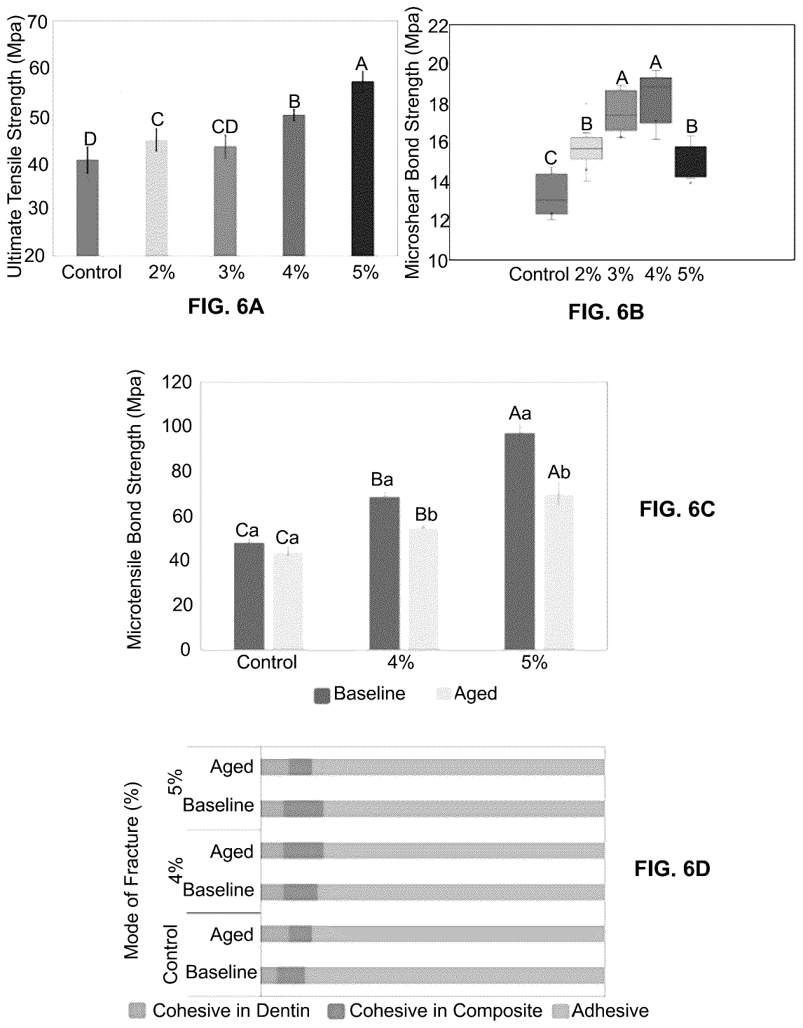

The ultimate tensile strength (UTS) test is a critical measure for assessing the robustness and endurance of dental adhesives, as it determines the maximum stress a material can withstand before rupture. In the present invention, the UTS values for the dental adhesives showed a positive correlation with the concentration of the active component, ranging from 44.6 (±2.3) MPa in the 2% group to 56.9 (±2.3) MPa in the 5% group, with the increase being statistically significant (p<0.05) as illustrated in FIG. 6A. The adhesives with 4% and 5% concentrations exhibited the highest UTS values. This enhancement in strength can be attributed to the higher content of CHX@SiQuac@Fe₃O₄@m-SiO₂ particles, which, by increasing the proportion of inorganic fillers, effectively reduces the relative amount of the organic matrix, thus reinforcing the overall polymer structure (67).

The micro-shear bond strength (μ-SBS) outcomes, as illustrated in FIG. 6B, demonstrate that incorporating CHX@SiQuac@Fe₃O₄@m-SiO₂ into the dental adhesive significantly enhanced the μ-SBS values by at least 11% in comparison to the control group (p<0.05). The peak μ-SBS results were observed in Groups with 2 and 3 wt. % adhesives achieved 17.5 (±0.9) MPa and 18.5 (±1.1) MPa, respectively. However, the adhesive formulation with the highest CHX@SiQuac@Fe₃O₄@m-SiO₂ particles exhibited lower μ-SBS values than the other concentrations (p<0.05). This decrease in bond strength at the highest concentration may be attributed to the increased viscosity, which could hinder the material's penetration through the collagen fibrils of the dentin (28).

In the micro-tensile bond strength (μ-TBS) testing, a model was used that simulates pulpal pressure in a real clinical scenario (68). Assessing the micro-tensile bond strength (μ-TBS) of dental adhesives immediately after application and over time is crucial for determining dental restorations' long-term effectiveness and reliability (69). The preparation steps for microtensile bond strength (μ-TBS) comprise 1) remotion of enamel and roots, 2) bonding procedure with pulpal pressure and magnetic field application, 3) restoration cut into beams, 4) beams fixing in metallic jigs, 5) beams under tension until fracture, and 6) fracture pattern analysis using a stereomicroscope. Immediate μ-TBS testing ensures the adhesive can withstand the stresses after its placement. At the same time, longitudinal assessments are essential to understand how the bond strength holds up against the daily mechanical stresses and environmental factors encountered inside the mouth over time. According to the previous results of the micro shear, the formulations containing CHX@SiQuac@Fe₃O₄@m-SiO₂ particles at 4 wt. % and 5 wt. % reported the best bonding performance. Based on that, immediate and longitudinal (after artificial aging) μ-SBS was performed on groups containing CHX@SiQuac@Fe₃O₄@m-SiO₂ particles at concentrations greater than 4 wt. %.

FIG. 6C illustrates a marked increase in bond strength for the dental adhesive group that incorporated CHX@SiQuac@Fe₃O₄@m-SiO₂ particles at a 5 wt. % concentration. At the initial measurement (baseline), this group showed a 52.3% enhancement in bond strength compared to the control group. This improvement was sustained even after aging, with the 5 wt. % group exhibiting approximately a 26% higher bond strength than the control (p<0.05). These findings indicate that adding particles at this concentration does not compromise the adhesive's ability to penetrate demineralized dentin and form a robust hybrid layer.

After aging, micro-tensile bond strength (μ-TBS) decreased by 66% and 50% for the 4% and 5% groups, respectively, in comparison to immediate (baseline) values. Despite this reduction, these groups outperformed the control group (p<0.05). This relative durability may be attributed to improved adhesive infiltration by the magnetic motion of particles into the dentin during the adhesive application, which could enhance hybridization and minimize the impact of simulated pulpal pressure on the bond strength (19). It has been hypothesized that magnetic particles might enhance resin infiltration at the bottom of the mixed layer, potentially leading to a superior hybrid layer formation (70). Furthermore, the magnetic field's force is thought to facilitate deeper penetration of Bis-GMA into the dentin, reducing phase separation between Bis-GMA and HEMA, which could subsequently strengthen the bond (71).

Fracture pattern analysis revealed that most fractures in all groups occurred at the adhesive interface (FIG. 6D) at both initial and aged time points. This indicates that the force applied during testing was appropriately perpendicular to the interface. These findings are consistent with reported literature (21), where stress distribution under tensile stress was evenly distributed across the adhesive interface.

Although significant, the observed decrease in micro-tensile bond strength (μ-TBS) after aging for both the 4% and 5% groups did not undermine the overall performance of these groups compared to the control. The fact that these groups with higher concentrations of CHX@SiQuac@Fe₃O₄@m-SiO₂ particles still showed improved bond strength relative to the control, even after the aging process, suggests that the incorporation of these particles may confer long-term benefits to the adhesive's structural integrity. This could be particularly relevant in the stressful environment of the oral cavity, where restorations are subjected to various mechanical and biochemical challenges over time.

Utilizing a simulated pulpal pressure model herein significantly enhances the clinical relevance of the present invention. This model closely mimics the actual conditions within a living tooth, where the pulpal fluids exert an inward pressure that can influence the penetration and adhesion of dental restoratives (72, 73). In the absence of simulated pulpal pressure, the outcomes related to the movement of superparamagnetic iron oxide nanoparticles (SPIONs) toward the pulp could be overstated and not reflective of the actual behavior inside a living tooth. Simulated pulpal pressure is crucial to emulate the pulp's natural inward fluid dynamics, which counteracts adhesive penetration. Without this counterpressure, the magnetic attraction of SPIONs towards the pulp chamber might appear more pronounced than it would be under normal physiological conditions, potentially skewing the results and compromising the study's clinical translation potential (74).

By employing both microshear and micro-tensile bond strength (μ-TBS) tests, the adhesive capabilities of the materials were assessed thoroughly. The μ-TBS is especially noteworthy as it provides a more sensitive measure of bond strength, allowing for the detection of subtle differences in adhesive performance that might not be apparent with traditional macro-shear tests (75). This enhanced discriminative power is crucial for identifying the best-performing adhesive formulations that can withstand the rigors of the oral environment.

Moreover, the temporal perspective given by the longitudinal assessment of bond strength adds a vital dimension. While immediate bond strength is important for initial clinical success, understanding how adhesives hold up over time is critical for predicting long-term performance (74). By evaluating bond strength after an aging period, insights were provided into the durability of adhesive bonds and their potential to maintain integrity over the life of a dental restoration.

Antibacterial & Cytotoxicity Performance of the Adhesives containing CHX@SiQuac@Fe₃O₄@m-SiO₂

Incorporating antibacterial and bioactive particles into dental resin formulations is critical to developing dental materials. However, the effectiveness of these particles can be significantly reduced when mixed with dental monomers due to interactions that may diminish their intended benefits (76). To address this, adhesives' antibacterial and cytotoxicity performance containing CHX@SiQuac@Fe₃O₄@m-SiO₂, were assessed ensuring their incorporation maintains the desired antibacterial efficacy.

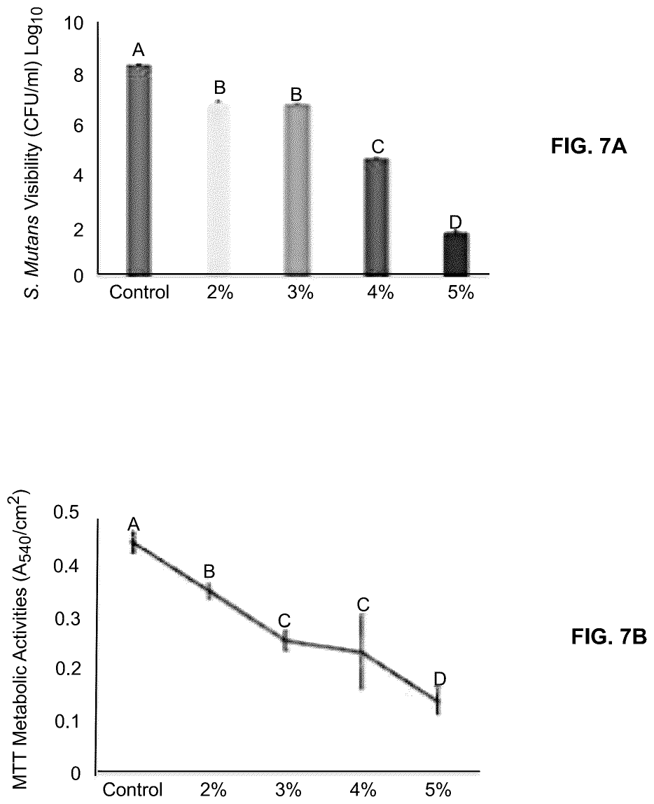

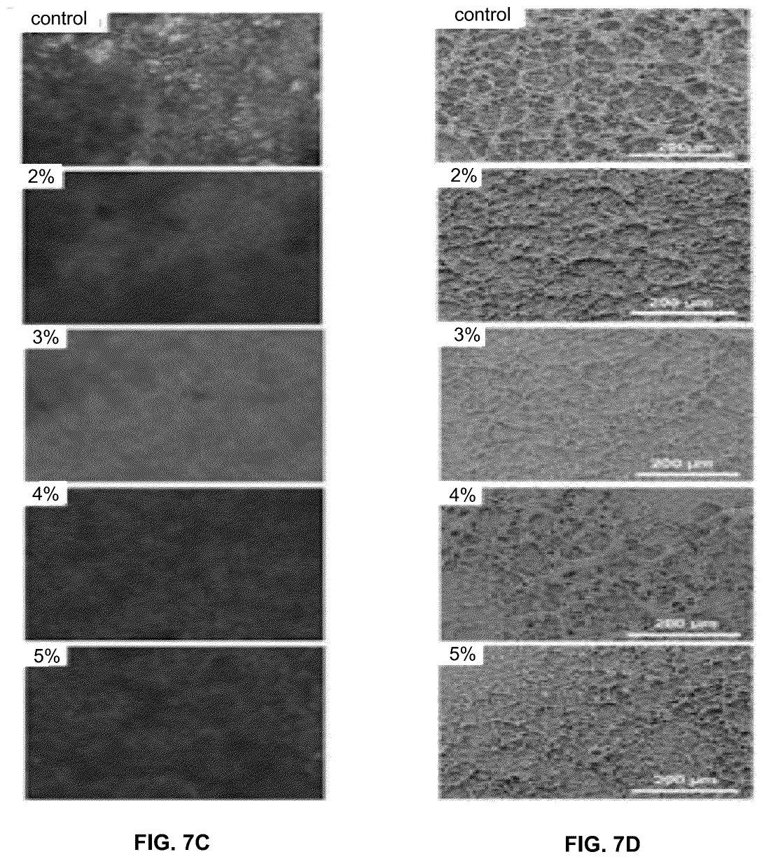

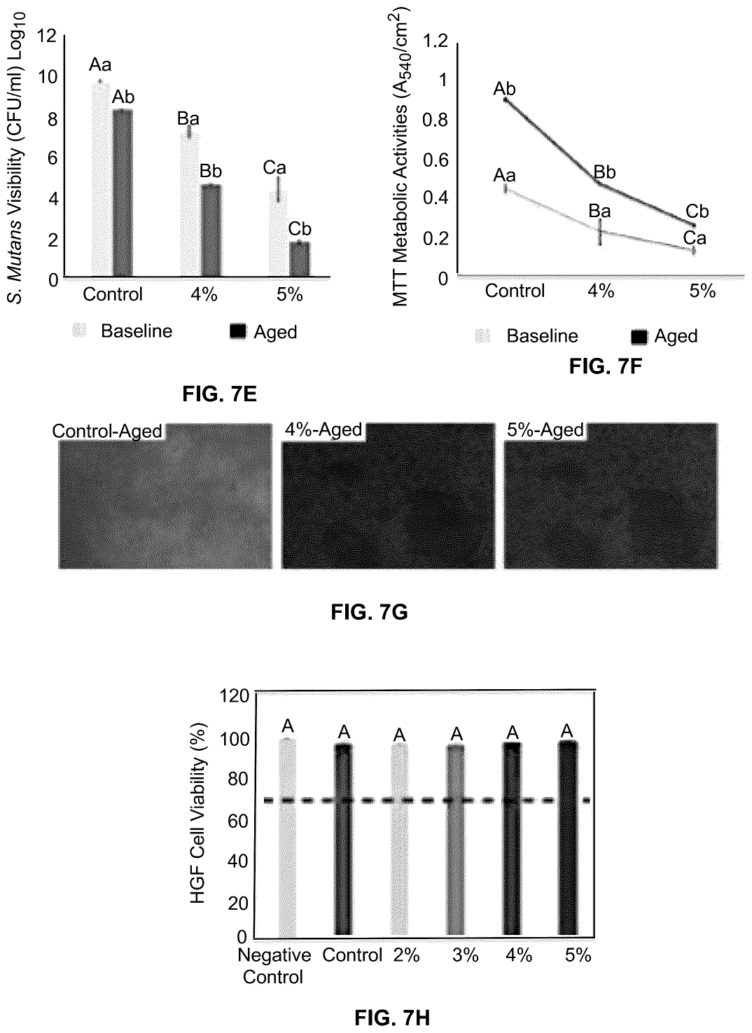

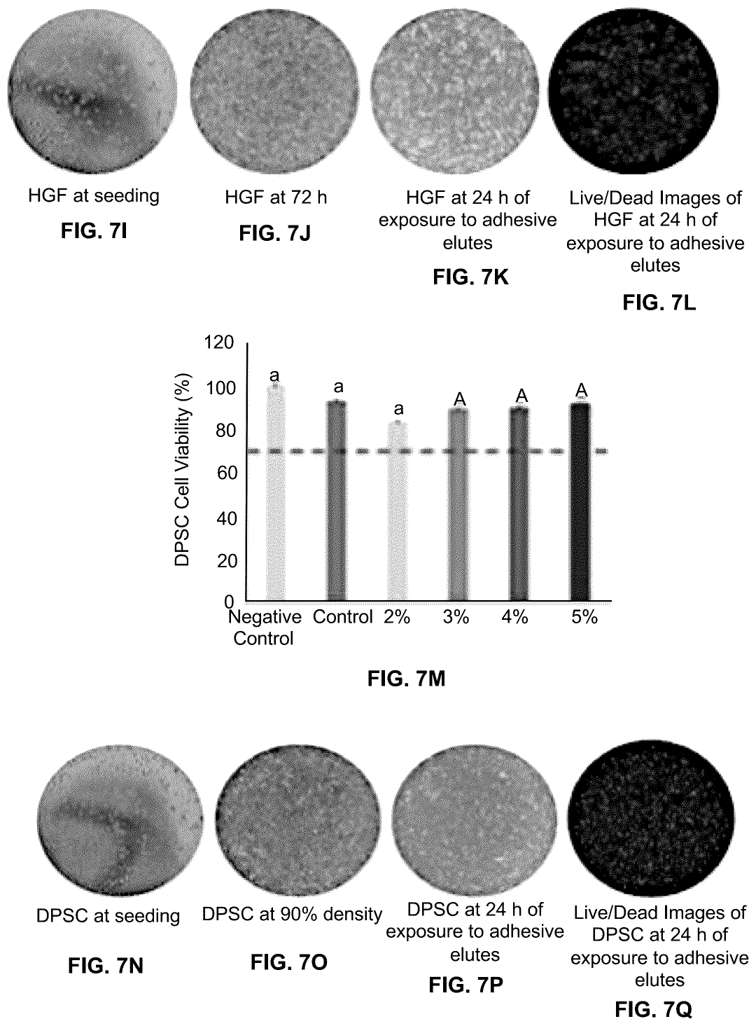

To ensure the long-term effectiveness and safety of the newly developed dental adhesives containing CHX@SiQuac@Fe₃O₄@m-SiO₂, immediate and long-term assessments of their antibacterial efficacy and cell viability were conducted, (FIGS. 7A-7L). The immediate biofilm assessment against S. mutans biofilms included a CFU assay and an evaluation of biofilm formation's metabolic activity on the adhesives. Adhesives formulated with CHX@SiQuac@Fe₃O₄@m-SiO₂ were included at the concentrations that had shown significant antibacterial effectiveness in earlier assays. For the long-term assessment conducted after artificial aging, those concentrations that had exhibited optimal performance in bond strength tests were examined to understand their durability and sustained antibacterial action over time.

The CFU analysis revealed a minimum of 17% reduction in bacterial growth of S. mutans biofilm for the 2% group compared to the control. The most significant decreases were observed adhesives formulated with CHX@SiQuac@Fe₃O₄@m-SiO₂ at 4 and 5 wt. %, with around 3 and 6 log reductions in bacterial count (FIG. 7A). Increasing the CHX@SiQuac@Fe₃O₄@m-SiO₂ mass fraction has shown concentration-dependence lower metabolic activity, with adhesives formulated with CHX@SiQuac@Fe₃O₄@m-SiO₂ at 5 wt. % presenting metabolic activity approx. 70% lower than control (p<0.05) (FIG. 7B).

FIG. 7C presents live/dead staining images of biofilms on control and four tested adhesive concentrations. In these images, live bacteria appear green, while dead bacteria are red; overlapping live and dead bacteria produce yellow or orange hues. The control adhesive showed a dense layer of predominantly live bacteria. In contrast, adhesives with 2 to 4 wt. % CHX@SiQuac@Fe₃O₄@m-SiO₂ significantly increased compromised (dead) bacteria. At 5 wt. % CHX@SiQuac@Fe₃O₄@m-SiO₂, most bacteria were compromised. The scanning electron microscopy (SEM) images suggest a disruption in the arrangement and distribution of biofilms over adhesives at 2 to 5 wt. % CHX@SiQuac@Fe₃O₄@m-SiO₂. These visual results align with and support the findings from the MTT and CFU assays.

After artificial aging, the antibacterial properties were maintained, with a more pronounced bacterial reduction observed for adhesives at 5 wt. % CHX@SiQuac@Fe₃O₄@m-SiO₂. The bacterial reduction was lower after aging, significantly different from the baseline results (FIG. 7E). The metabolic activity followed a similar trend and decreased by approximately. 50% after aging (FIG. 7F), with live/dead images showing more compromised bacteria (red color) than in the control group (FIG. 7G). Cytotoxicity was evaluated against human gingival fibroblasts (HGF) and dental pulp stem cells (DPSC). The live/dead images demonstrated high cell viability across all groups, with cell viability values above 70% and no significant difference from the control group (FIG. 7H-7Q).

The results following artificial aging of the dental adhesives containing 5 wt. % of CHX@SiQuac@Fe₃O₄@m-SiO₂ demonstrate their enduring antibacterial properties. Notably, even after aging, these adhesives showed a significant bacterial reduction compared to the baseline results. This finding is critical as it suggests the durability of the antibacterial effectiveness over time, a key factor for long-term clinical success in dental restorative treatment. The antibacterial activity of CHX@SiQuac@Fe₃O₄@m-SiO₂-containing adhesives is mainly attributed to the release of CHX and the positive charge density of the quaternary ammonium segment in the SiQuac structure without the release of these methacrylates derived from quaternary ammonium salts (13, 34, 35). Here, the material was challenged with an artificial aging proposed to simulate six months of material behavior inside the mouth. Factors such as leaching of the active ingredients, breakdown of the polymer matrix, and loss of mechanical integrity may contribute to a reduction in the antibacterial effectiveness of the material over time. Despite these challenges, the sustained antibacterial effect observed herein post-aging is noteworthy. The retention of antibacterial activity in the CHX@SiQuac@Fe₃O₄@m-SiO₂-containing adhesives, even after being subjected to artificial aging, demonstrates their potential durability and effectiveness in a clinical setting.

Regarding quaternary ammonium-based components, it's important to note that not all materials containing these compounds maintain their antibacterial effectiveness over an extended period (77). The persistence of this effect largely depends on the ability of the antibacterial monomer to cross-polymerize effectively within the dental material (78). In the case of the SiQuac silanized particles used herein, the chemical bonding of SiQuac to the shell likely contributes to the retention of the antibacterial properties even after aging.

### REFERENCES

1. Feitosa, et al. J Adhes Dent 2019, 21(3):199-207.
2. Mokeem, et al. Biomedicines 2023, 11(5):1256.
3. Perdigão, et al. Jpn Dent Sci Rev 2020, 56(1):190-207.
4. Shah et al. J Conserv Dent 2021, 24(5):415-420.
5. Li, et al. Dent Mater 2018, 34(9):1310-1322.
6. Ji, et al. Nanomedicine 2018, 14(3):919-927.
7. Higino, et al. Biomed Phys Eng Express 2022, 8(4).
8. Jahns, et al. Journal of Materials Chemistry B 2020, 8(4):776-786.
9. Boutsiouki, et al. J Adhes Dent 2023, 25(1):13-22.
10. del Hierro, et al. Molecular Catalysis 2018, 450:112-120.
11. Cui, et al. Magnetochemistry 2023, 9(1):1.
12. Bapat, et al. Front Microbiol 2022, 13:927282.
13. Daood, et al. Sci Rep 2020, 10(1):10970.
14. Pellas, et al. ACS Appl. Nano Mater. 2021, 4(9):9842-9854.
15. Priyadarshini, et al. Dent Mater 2017, 33(7):830-846.
16. Sideridou, et al. Dent Mater 2009, 25(11):1315-1324.
17. Akram, et al. J Nanobiotechnology 2021, 19(1):43.
18. Abedi, et al. J Nanobiotechnology 2021, 19(1):18.
19. Salvador, et al. Computational Biology and Chemistry 2017, 71:82-88.
20. Tezvergil-Mutluay, et al. J Dent Res 2011, 90(4):535-540.
21. Garcia, et al. J Dent 2020, 102:103477.
22. Mokeem, et al. J Funct Biomater 2022, 13(4):190.
23. Li, et al. Dent Mater 2014, 30 (4):433-441.
24. Antonucci, et al. Dent Mater 2012, 28(2):219-228.
25. 14:00-17:00. ISO 4049:2009.
26. Collares, et al. J Adhes Dent 2011, 13(2):125-129.
27. Garcia, et al. J Dent Res 2016, 95(12):1401-1407.
28. Garcia, et al. Acta Biomater 2021, 134:337-347.
29. Tian, et al. Acta Biomater 2016, 38:190-200.
30. Ibrahim, et al. Int J Mol Sci 2019, 20(14):3491.
31. Zhang, et al. Dent Mater 2015, 31(9):1119-1131.
32. 4:00-17:00. ISO/TS 11405:2015.
33. Sigusch, et al. Dent Mater 2012, 28(3):312-319.
34. Gale, et al. J Dent 1999, 27(2):89-99.
35. Trzeciak, et al. Pharmaceutics 2021, 13(7):950.
36. Sajid, et al. Progress in Natural Science: Materials International 2023, 33(1):116-119.
37. Millot, et al. J. Am. Chem. Soc. 2023, 145(12):6671-6681.
38. Grisolia, et al. Molecules 2023, 28(13), 5105.
39. Sing, K. Colloids and Surfaces A: Physicochemical and Engineering Aspects 2001, 187-188:3-9.
40. Vazquez, et al. Boletín de la Sociedad Española de Cerámica y Vidrio 2017, 56(3):139-145.
41. Vieira, et al. Colloids Surf B Biointerfaces 2019, 174:224-231.
42. Xu, et al. Bulletin of the Korean Chemical Society 2013, 34.
43 2. Balhaddad, et al. ACS Nano 2021.
44. Nguyen, et al. Applied Sciences 2021, 11(23):11301.
45. Priyadarshini, et al. Bioengineering (Basel) 2017, 4(3):59.
46. Azadpour, et al. Materials (Basel) 2022, 15(24):8762.
47. Havlíková, L et al. Journal of Pharmaceutical and Biomedical Analysis 2007, 43(3):1169-1173.
48. de Moraes, et al. Restor Dent Endod 2020, 45(3):e22225.e31.
49. Abakumov, et al. J Biochem Mol Toxicol 2018, 32(12):e22225.
50. e Souza, et al. Am J Dent 2007, 20(6):400-404.
51. Jiao, et al. Progress in Polymer Science 2017, 71:53-90.
52. 14:00-17:00. ISO 10993-5:2009.
53. Jakubovics, et al. Periodontology 2000 2021, 86(1):32-56.
54. Shree, et al. Medicine in Microecology 2023, 16:100084.
55. iller, et al. Proc Natl Acad Sci U S A 2001, 98(11):5981-5985.
56. Tichy, et al. Oper Dent 2020, 45(5):556-566.
57. Maktabi, et al. Am J Dent 2018, 31(6):320-328.
58. Maktabi, et al. J Dent 2019, 88:103110.
59. Huang, et al. Acta Biomater 2018, 81:158-168.
60. Law, et al. Pure and Applied Chemistry 2015, 87(8):759-765.
61. Balhaddad, et al. Dent Mater 2020, 36(8):e266-e278.
62. Namba, et al. Dent Mater 2009, 25(4):424-430.
63. Murata, et al. Biomaterials 2007, 28(32):4870-4879.
64. Wang et al. Dent Mater 2019, 35(6) :: 847-861
65. Santerre, et al. Crit Rev Oral Biol Med 2001, 12(2):136-151.
66. Ferracane, J. L. Dent Mater 2006, 22(3):211-222.
67. Lohbauer, et al. Acta Biomater 2010, 6(12):4539-4546.
68. Silva, et al. International Journal of Adhesion and Adhesives 2022, 116:103139.
69. Sano, et al. Jpn Dent Sci Rev 2020, 56(1):24-31.
70. Rahal, et al. Acta Odontol Latinoam 2011, 24(1):8-14
71. Pashley, et al. Dent Mater 2011, 27(1):1-16.
72. Cardoso, et al. Braz. oral res. 2008, 22(2):170-175.
73. Feitosa, et al. J Dent 2012, 40(12):1134-1143.
74. Van Meerbeek, et al. Dent Mater 2010, 26(2):e100-121.
75. Sano, et al. Dent Mater 1994, 10(4):236-240.
76. Melo, et al. J Dent Res 2023, 102(11):1180-1190.
77. Bento de Carvalho, et al. Biology 2023, 12(5):669.
78. Mokeem, et al. J Funct Biomater 2022, 13(4):190.
79. Pietrella, et al. ACS Biomater. Sci. Eng. 2018, 4(12):4144-4153.
80. Gou, et al. Dent Mater 2018, 34(12):1814-1827.
81. Morel, et al. ACS Nano 2008, 2(5):847-856.
82. 14:00-17:00. ISO/TS 11405:2015.

What is claimed:

1. A biomedical composition for dental restoration, comprising:
a plurality of magnetic microparticles;
an antibiotic; and
a mesoporous silica encapsulating the plurality of magnetic microparticles and the antibiotic with an antibacterial shell disposed therearound.

2. The composition of claim 1, wherein the plurality of magnetic microparticles comprise iron oxide Fe₃O₄.

3. The composition of claim 1, wherein the antibiotic is chlorhexidine.

4. The composition of claim 1, wherein the antibacterial shell is an antibacterial quaternary ammonium silane.

5. The composition of claim 1, wherein the biomedical composition has a formula CHX@SiQuac@Fe₃O₄@m-SiO₂.

6. A dental formulation for a dental bonding restoration, comprising:
a dental adhesive material; and
the biomedical composition of claim 1 incorporated therein.

7. A method for increasing longevity of a dental restoration in a subject, comprising:
preparing the dental formulation of claim 6, applying the dental formulation to an area of interest in an oral cavity of the subject; and
bonding the dental formulation to dentin in the area of interest, wherein the magnetic microparticles, the antibiotic and the antibacterial shell of the biomedical composition incorporated therein increase the longevity of the dental restoration in the subject.

8. The method of claim 7, wherein the biomedical composition strengthens an interfacial bond between the dental adhesive and the dentin.

9. A dental adhesive, comprising:
a biomedical adhesive material; and
a magnetic core-shell platform incorporated therein, said dental adhesive pharmacologically effective for a tooth restoration.

10. The dental adhesive of claim 9, wherein the magnetic core-shell platform comprises a core of a plurality of magnetic microparticles doped with an antibiotic and coated with a mesoporous silane shell that is functionalized with an antibacterial agent.

11. The dental adhesive of claim 10, wherein the plurality of magnetic microparticles are iron oxide Fe₃O₄.

12. The dental adhesive of claim 10, wherein the plurality of magnetic microparticles are doped with chlorhexidine.

13. The dental adhesive of claim 10, wherein the antibacterial agent is an antibacterial quaternary ammonium silane.

14. The dental adhesive of claim 9, wherein the magnetic core-shell platform has a formula of CHX@SiQuac@Fe₃O₄@m-SiO₂.

15. A method for restoring a tooth in a subject in need thereof, comprising:
formulating a dental adhesive comprising a dental adhesive material and containing a multifunctional magnetic platform therein;
preparing the tooth for restoration;
applying the dental adhesive to a dentin layer on the tooth; and
restoring the tooth.

16. The method of claim 15, wherein the multifunctional magnetic platform has a core-shell structure comprising:
a core formed of a plurality of magnetic microparticles and an antibiotic doping agent; and
an antibacterial silane shell coating the core.

17. The method of claim 16, wherein the core is formed of a plurality of iron oxide Fe₃O₄ microparticles and doped with chlorhexidine.

18. The method of claim 16, wherein the antibacterial silane shell is a mesoporous silane comprising an antibacterial quaternary ammonium silane.

19. The method of claim 16, wherein the multifunctional magnetic platform has a core-shell structure of CHX@SiQuac@Fe₃O₄@m-SiO₂.

20. The method of claim 15, wherein the multifunctional magnetic platform contained in the dental adhesive upon forming an interfacial hybrid bond with the dentin layer during restoration increases longevity thereof via:
increasing bonding stability via magnetic interactions;
resisting degradation of the hybrid bond by matrix metalloproteinases; or inhibiting bacterial growth; or a combination thereof.
